(19) United States
(12) Patent Application Publication
(10) Pub. No.: US 2007/0219268 A1
(43) Pub. Date: Sep. 20, 2007

(54) Anti-cancer activity augmentation compounds and formulations and methods of use thereof

(75) Inventor: Frederick H. Hausheer, Boerne, TX (US)

Correspondence Address: Scott A. Whitaker, Ph.D., J.D. -Sr.;Intellectual Property Counsel, BioNumerik Pharmaceuticals, Inc., 8122 Datapoint Drive, Suite 1250, San Antonio, TX, 78229, US

(73) Assignee: BioNumerik Pharmaceuticals, Inc.

(21) Appl. No.: 11/724,933

(22) Filed: Mar. 16, 2007

Related U.S. Application Data
(60) Provisional application No. 60/782,826, filed on Mar. 16, 2006.

(51) Int. Cl. A61K 31/255; A61K 31/095

(52) U.S. Cl. 514/517; 514/706; 604/890.1

(57) ABSTRACT

The field of the present invention comprises pharmaceuticals and pharmaceutical treatments, including, for example, (i) compounds and formulations which cause the augmentation of anti-cancer activity (i.e., by enhancement of the lethal cytotoxic action in stimulatory [inducing oxidative stress] and/or depletive [decreasing anti-oxidative capacity] manner) of chemotherapeutic agents, in a selective manner; (ii) methods of administering said anti-cancer augmentation compounds and formulations; (iii) delivery devices containing said anti-cancer augmentation compounds and formulations; and (iv) methods of using said anti-cancer augmentation compounds, formulations, and devices to treat subjects in need thereof.

### RELATED APPLICATIONS

The present application claims priority to Provisional Application Ser. No. 60/782,826 filed Mar. 16, 2006 and entitled: “ANTI-CANCER ACTIVITY AUGMENTATION COMPOUNDS AND FORMULATIONS AND METHODS OF USE THEREOF”

### FIELD OF THE INVENTION

The field of the present invention relates to pharmaceuticals and pharmaceutical treatments, including, for example, compounds and formulations which cause augmentation of anti-cancer activity by enhancement of the lethal cytotoxic action of chemotherapeutic agents, as well as methods of administering said compounds and formulations which cause augmentation of the anti-cancer activity of chemotherapeutic agents to subjects in need thereof. The present invention also relates to devices for the administration of said compounds and formulations to treat subjects in need thereof.

### BACKGROUND OF THE INVENTION

As the number of agents and treatments for cancer, as well as the number of subjects receiving one or more of these chemotherapeutic agents concomitantly, has increased, clinicians and researchers are fervently seeking to fully elucidate the biological, chemical pharmacological, and cellular mechanisms which are responsible for the pathogenesis and pathophysiology of the various adverse disease manifestations, as well as how these chemotherapeutic drugs exert their anti-cancer and cytotoxic activity on a biochemical and pharmacological basis. Unfortunately, however, there is no treatment presently available which is generally safe and effective for augmenting the anti-cancer activity of chemotherapeutic agents for either preventing or delaying the initial onset of, attenuating the overall severity of, and/or expediting the resolution of the acute and/or chronic pathophysiology associated with malignancy. The potential pathophysiological mechanisms responsible for such these aforementioned manifestations are not fully known, and in many cases are topics of energetic debate. Furthermore, as described herein, with the exception of the novel conception and practice of this invention, there are no agents currently approved for the enhancement of tumor cell kill or augmented cytotoxicity on cancer cells in a selective manner while avoiding deleterious chemotherapeutic agent-induced effects on normal (i.e., non-cancerous) cells and tissues.

In brief, the present invention discloses and claims: (i) compounds and formulations which cause augmentation of the anti-cancer activity of chemotherapeutic agents (i.e., enhancement of the cytotoxic action of chemotherapy treatment in a stimulatory [inducing oxidative stress] and/or a depletive [decreasing anti-oxidative capacity] manner) by increasing intracellular oxidative stress within cancer cells in a selective manner while avoiding deleterious chemotherapeutic agent-induced effects on normal (i.e., non-cancerous) cells and tissues; (ii) methods of administering said compounds and formulations which augment the anti-cancer activity of chemotherapeutic agents; (iii) delivery devices which contain and administer said compounds and formulations which augment the anti-cancer activity of chemotherapeutic agents; and (iv) methods of using said compounds, formulations, and devices which augment the anti-cancer activity of chemotherapeutic agents to treat subjects in need thereof. The compounds and formulations of the present invention comprise an effective amount of a 2,2′-dithio-bis-ethane sulfonate, a pharmaceutically-acceptable salt thereof, and/or an analog thereof, which also include the compounds of Formula (I). The compounds of Formula (I) include pharmaceutically-acceptable salts of such compounds, as well as prodrugs, analogs, conjugates, hydrates, solvates and polymorphs, as well as stereoisomers (including diastereoisomers and enantiomers) and tautomers of such compounds. Compounds of Formula (I), and their synthesis are described in published U.S. Patent Application No. 2005/0256055, the disclosure of which is hereby incorporated by reference in its entirety. It should be noted that all of the aforementioned chemical entities in the previous three (3) sentences are included in the terms “a dithio-containing compound of the present invention”, “dithio-containing compounds of the present invention”, or “dithio-containing compound(s),” as utilized herein, unless otherwise specifically stated, including the metabolite of disodium 2,2′-dithio-bis-ethane sulfonate, known as 2-mercapto ethane sulfonate sodium. 2-mercapto ethane sulfonate sodium is also known in the literature as mesna. The disodium salt of 2,2′-dithio-bis-ethane sulfonate has also been referred to in the literature as dimesna, Tavocept™, and BNP7787.

The mechanisms of the dithio-containing compounds of the present invention in the augmentation of the anti-cancer activity of chemotherapeutic agents may involve one or more of several novel pharmacological and physiological factors, including but not limited to, a prevention, compromise and/or reduction in the normal increase, responsiveness, or in the concentration and/or tumor protective metabolism of glutathione/cysteine and other physiological cellular thiols; these antioxidants and enzymes are increased in concentration and/or activity, respectively, in response to the induction of intracellular oxidative stress which may be caused by exposure to cytotoxic chemotherapeutic agents in tumor cells. The dithio-containing compounds of the present invention may exert an oxidative activity by the intrinsic composition of the molecule itself (i.e., an oxidized disulfide), as well as by oxidizing free thiols to form oxidized disulfides (i.e., by non-enzymatic SN2-mediated reactions, wherein attack of a thiol/thiolate upon a disulfide leads to the scission of the former disulfide which is accompanied by the facile departure of a thiol-containing group. As the thiolate group is far more nucleophilic than the corresponding thiol, the attack is believed to be via the thiolate, however, in some cases the sulfur atom contained within an attacking free sulfhydryl group may be the nucleophile), and may thereby lead to pharmacological depletion and metabolism of reductive physiological free thiols (e.g., glutathione, cysteine, and homocysteine). The Applicant has determined that some of the novel principles governing these reactions involve the increased (i.e., greater stability of) solvation free energy of the new disulfide and free thiol products that are formed from the reaction; therefore these reactions appear to be largely driven by the favorable thermodynamics of product formation (i.e., exothermic reactions). One or more of these pharmacological activities will thus have an augmenting (additive or synergistic) effect on the cytotoxic activity of chemotherapeutic agents administered to patients with cancer, with the additional cytotoxic activity resulting from the combined administration of the dithio-containing compounds of the present invention and chemotherapy compounds, thereby leading to: (i) an increase in the cytotoxic and cytoreductive anti-cancer efficacy and decreases in tumor-mediated resistance of the various co-administered chemotherapeutic agents, e.g., platinum- and alkylating agent-based drug efficacy and tumor-mediated drug resistance; (ii) thioredoxin inactivation by the dithio-containing compounds of the present invention, thereby increasing apoptotic sensitivity and decreasing mitogenic/cellular replication signaling in cancer cells; (iii) the killing of cancer cells directly by a key metabolite of disodium 2,2′-dithio-bis-ethane sulfonate (also known in the literature as dimesna, Tavocept™, or BNP7787), 2-mercapto ethane sulfonate sodium (also known in the literature as mesna) which possesses intrinsic cytotoxic activity (i.e., causes apoptosis) in some tumors by an, as yet, unknown mechanism; and/or (iv) 2,2′-dithio-bis-ethane sulfonate compounds (and possibly mesna) acting to enhance oxidative stress or compromise the anti-oxidative response of cancerous tumor cells, or both, which may thereby enhance their oxidative biological and physiological state. This may serve to subsequently increase the amount of oxidative damage (e.g., as mediated by reactive oxygen species (ROS), reactive nitrogen species (RNS), or other mechanisms) in tumor cells exposed to chemotherapy, thereby enhancing cytotoxicity/apoptosis of chemotherapy agents. Thus, by enhancing oxidative stress and/or reducing or compromising the total anti-oxidative capacity or responsiveness of cancer tumor cells, an increase in anti-cancer activity can be achieved. It is believed by the Applicant of the present invention that this is a key mechanism of action in the augmentation of the anti-cancer activity of chemotherapeutic agents that may act in concert with the other aforementioned mechanisms of augmentation of the anti-cancer activity mediated by the dithio-containing compounds of the present invention and metabolites thereof (e.g., 2-mercapto ethane sulfonate). This has extremely important implications for the treatment of cancer.

Compositions and formulations comprising the dithio-containing compounds of the present invention may either be given: (i) in a stimulatory (i.e., inducing oxidative stress) and/or depletive (i.e., decreasing anti-oxidative capacity or responsiveness) manner to a cancer patient prior to the administration of an oxidative stress-inducing chemotherapeutic agent or agents in order to sensitize the neoplasm to enhance the tumor cytotoxicity of the chemotherapeutic agent or agents; (ii) in a therapeutic manner, as a cancer patient begins a chemotherapy cycle, in order to augment the activity of the oxidative stress induced by the chemotherapeutic agent or agents; and/or (iii) in a subsequent manner (i.e., after the chemotherapy cycle) in order to continue the induction or maintenance of the oxidative stress process in cancer cells. Additionally, the aforementioned compositions and formulations may be given in an identical manner to augment or enhance the anti-cancer activity of a cytotoxic agent by any clinically-beneficial mechanism(s).

### I. Cellular Response to Oxidative Stress

The formation of potentially physiologically-deleterious reactive oxygen species (ROS) and that of reactive nitrogen species (RNS), may be caused from a variety of metabolic and/or environmental processes. By way of non-limiting example, intracellular ROS (e.g., hydrogen peroxide: H₂O₂; superoxide anion: O₂⁻; hydroxyl radical: OH⁻; nitric oxide: NO; and the like) may be generated by several mechanisms: (i) by the activity of radiation, both exciting (e.g., UV-rays) and ionizing (e.g., X-rays); (ii) during xenobiotic and drug metabolism; and (iii) under relative hypoxic, ischemic and catabolic metabolic conditions, as well as by exposure to hyperbaric oxygen. The electron transport chain localized in the smooth endoplasmic reticulum and mitochondria operates to hydroxylate different substrates (e.g., steroids, drugs, carcinogens, and other lipid-soluble species) to render them more hydrophilic and, hence, more easily removable. With regard to the electron transport chain, O₂⁻ may be generated by the “leakage” of electrons from NADPH cytochrome P450 reductase and by the release from cytochrome P450 during substrate hydroxylation. The electron transport chain of the mitochondria is also a well-documented source of H₂O₂ from disproportionate O₂⁻ production. There is also an additional mitochondrial source of ROS, not linked to respiration and located in the outer mitochondrial membrane, for example, monoamine oxidase deamination of biologic amines is associated with a large production of H₂O₂. In cellular respiration, molecular oxygen is normally reduced to water through the mitochondrial respiratory chain in an extremely efficient manner, however, approximately 1-2% of the electrons may be “leaked” and generate O₂⁻ by the action of coenzyme Q (i.e., ubiquinone) and a reduced component of NADH dehydrogenase. See, e.g., Georgiou, G. How to Flip the (Redox) Switch. Cell 111:607-610(2002).

The reactive nitrogen species (RNS) of current interest in causing oxidative stress include oxides of nitrogen, nitrogen dioxide (NO₂) and nitric oxide (NO). Nitric oxide is produced by the vascular endothelium and other cells in the body from the amino acid L-arginine. Nitric oxide is believed to be poorly reactive with most molecules within the human body (non-radicals) but, as a free radical, it can react extremely rapidly with other free radicals (e.g., superoxide, amino acid radicals, and certain transition metal ions). The reaction between nitric oxide and superoxide produces peroxynitrite (ONOO⁻), which can be a highly reactive species.

Protection against the harmful physiological activity of ROS and RNS species is reported to be mediated by a complex network of overlapping mechanisms that utilize a combination of small redox-active molecules and enzymes coupled with the expenditure of reducing equivalents. Oxidative stress occurs when the rate of generation of reactive compounds exceeds the cellular detoxification capacity of such reactive compounds (e.g., ROS and RNS) of the cells. Perhaps one of the most widely-studied harmful ROS-mediated and RNS-mediated phenomena is that of the aberrant modification of protein thiols. The accumulation of oxidized or nitrosylated cysteines in proteins has detrimental consequences for cellular function and results in a condition generally, albeit somewhat imprecisely, described as “redox stress”. In brief, it is the production of redox-mediated modification of cellular proteins that confers a response to ROS and RNS species, with concomitant changes in redox status that regulate the initiation of signal transduction pathways and the induction of gene expression. These redox stress-mediated cellular responses generally involve the activation of genes involved in the detoxification of the ROS and RNS molecules and in the repair of any damage caused by their activity. Also, ROS and RNS may damage both single-stranded and double-stranded DNA by reactions with the phosphodiester/phosphate backbone, thereby leading to DNA fragmentation and cellular toxicity. In addition, ROS may result in the peroxidation of lipids (e.g., the formation of epoxides), thereby resulting in deleterious activity on cellular membrane stability, integrity and function.

The redox state of any particular biological environment can be defined as the sum of oxidative and reductive processes occurring within that environment which, in turn, directly relates to the extent to which molecules are oxidized or reduced within it. The redox potential of biological ions or molecules is a measure of their tendency to lose an electron (i.e., thereby becoming oxidized) and is expressed as E₀ in volts. The more strongly reducing an ion or molecule, the more negative its E₀. As previously stated, under normal physiological circumstances, most intracellular biological systems are predominantly found in a reduced state. Within cells, thiols (R—SH) such as glutathione (GSH), cysteine, homocysteine, and the like, are maintained in their reduced state, as are the nicotinamide nucleotide coenzymes NADH and NADPH. The opposite relationship is found in plasma, where the high partial pressure of oxygen (pO₂) promotes an oxidative environment, thereby leading to a high proportion (i.e., greater than 90%) of the physiological sulfur-containing amino acids and peptides (e.g., glutathione (GSH)) existing in stable oxidized (disulfide) forms. In plasma, there are currently no known enzymes that appear to reduce the disulfide forms of these sulfur-containing amino acids and GSH; this further contributes to the plasma vs. cellular disparity in terms of the relative proportions of physiological disulfides vs. thiols. Physiological circumstances can, however, arise which alter the overall redox balance and lead to a more oxidizing environment in the cell. In biological systems, this activity arises as a result of oxidative stress and physiological systems have evolved to remove, repair, and control the normal reducing environment. However, when oxidative stress overwhelms these protective mechanisms, oxidative damage and profound biological and toxic activity can occur.

Traditionally, both ROS and RNS have been considered only as deleterious and toxic substances involved in tissue injury, ischemia/low tissue perfusion, or hypoxic conditions, or under hyperbaric or high ambient pO₂ conditions. For example, the accumulation of ROS and RNS within non-phagocytic cells has been regarded as an unwanted by-product of oxidative phosphorylation, lipid metabolism, drug metabolism, ionizing radiation, and the like. Concentrations of ROS and RNS which cannot be adequately dealt with by the endogenous antioxidant system can lead to damage of lipids, proteins, carbohydrates, and nucleic acids.

The oxidative modification of these aforementioned biological molecules by toxic concentrations of ROS and RNS can lead to deleterious physiological consequences such as complete loss of function. It should be noted that while both ROS and RNS are involved in deleterious physiological and pathological processes, ROS have been more widely studied.

Recently, a new role for ROS has been proposed from the demonstration that ROS are capable of modifying both the structure and function of proteins. The production of sub-lethal concentrations of ROS has been shown to lead to alterations in both the intracellular and extracellular redox state, and it is such alterations that have been demonstrated to signal changes in cellular functions, thus contributing to the modulation of cell viability. This provides a means to regulate signal transduction pathways and gene expression, hence controlling a variety of cellular processes, which include, but are not limited to, induction and maintenance of the transformed state, programmed cell death (i.e., apoptosis) and cellular senescence, oxidative stress, and response to various drugs, growth factors, and hormones. It has now been established that, at concentrations compatible with normal cellular physiology, ROS appear able to exert a large variety of biochemical activities which may contribute to the modulation of cellular viability and function. Moreover, apart from their role in cellular signaling, ROS may also indirectly modulate cell function through the intervention of discrete amounts of products of their reaction(s) with defined biomolecules including, but not limited to, proteins, DNA, RNA, and lipids. In this relationship, an ever increasing amount of experimental data strongly supports the involvement of lipid oxidation products in cell signaling under both physiological and/or pathophysiological conditions. See, e.g., Martin, K. R. and Barrett, J. C., Human Exp. Toxicol. 21:71-76 (2002).

Cells, including cancer cells, can respond to oxidative stress by decreasing the levels of oxidants, such as ROS and oxidized thiols, as well as by the production of increased concentrations of free thiols and anti-oxidants. For example, superoxide anions are converted to H₂O₂ and O₂ by superoxide dimutase; whereas catalase, glutathione peroxidases, and peroxiredoxins reduce and detoxify such peroxides. Thiol reductases (e.g., thioredoxin and glutaredoxin) reduce disulfide bonds within proteins and oxidized thiol-based reductants.

Finally, molecular chaperones are also stimulated to mediate the refolding of unfolded and aggregated proteins. The genes encoding a variety of molecular chaperones, and proteins that catalyze ROS and disulfide bond metabolism are induced in response to oxidative stress. Elucidation of the mechanisms underlying such gene induction exemplified some of the earliest demonstrations of the specific modifications of proteins by ROS being part of defined biological processes. In addition to the activation of molecular chaperones by gene induction, there are now a growing number of examples of molecular chaperones that are activated directly by oxidative stress. It has also recently been recognized that cancer cells may respond to oxidative stress from chemotherapy and radiation exposure by decreasing the concentrations of ROS and oxidized thiols and well as by increased concentrations of thiol and anti-oxidants; when either or both of these mechanisms are operative, the subject's tumor cells may be resistant to chemotherapy and radiation therapy, thereby representing an important obstacle to curing or controlling the progression of the subject's cancer.

### II. Physiological Cellular Thiols

Thiol groups are those which contain functional CH₂—SH groups within conserved cysteinyl residues. It is these thiol-containing proteins which have been elucidated to play the primary role in redox-sensitive reactions. Their redox-sensing abilities are thought to occur by electron flow through the sulfhydryl side-chain. Thus, it is the unique properties afforded by the sulfur-based chemistry in protein cysteines (in some cases, possibly in conjunction with chelated central metal atoms) that is exploited by transcription factors which “switch” between an inactive and active state in response to elevated concentrations of ROS and/or RNS. It should be noted that the majority of cellular protein thiols are compartmentalized within highly reducing environments and are therefore “protected” from such oxidation. Hence, only proteins with accessible thiol groups, and high oxidation potentials are likely to be involved in redox-sensitive signaling mechanisms.

There are numerous naturally-occurring thiols and disulfides. The most abundant biologically-occurring amino acid is cysteine, along with its disulfide form, cystine. Another important and highly abundant intracellular thiol is glutathione (GSH), which is a tripeptide comprised of γ-glutamate-cysteine-glycine. Thiols can also be formed in those amino acids which contain cysteine residues including, but not limited to, cystathionine, taurine, and homocysteine. Many oxidoreductases and transferases rely upon cysteine residues for their physiological catalytic functions. There are also a large number of low molecular weight cysteine-containing compounds, such a Co-enzyme A and glutathione, which are vital enzymes in maintaining oxidative/reductive homeostasis in cellular metabolism. These compounds may also be classified as non-protein sulfhydryls (NPSH).

Structural and biochemical data has also demonstrated that thiol-containing cysteine residues and the disulfide cystine, play a ubiquitous role in allowing proteins to respond to ROS. The redox-sensitivity of specific cysteine residues imparts specificity to ROS-mediated cellular signaling. By reacting with ROS, cysteine residues function as “detectors” of redox status; whereas the consequent chemical change in the oxidized cysteine can be converted into a protein conformational change, hence providing an activity or response.

Within biological systems, thiols undergo a reversible oxidation/reduction reaction, as illustrated below, which are often catalyzed by transition metals. These reactions can also involve free radicals (e.g., thioyl RS) as intermediates. In addition, proteins which possess SH/SS groups can interact with the reduced form of GSH in a thiol-disulfide exchange. Thiols and their disulfides are reversibly linked, via specific enzymes, to the oxidation and reduction of NADP and NADPH, as shown in Table 1.

TABLE 1
Reversible Oxidation/Reduction Reaction

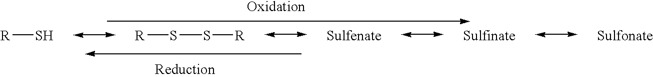

There is increasing experimental evidence that indicates that thiol-containing proteins are sensitive to thiol modification and oxidation when exposed to changes in the redox state. This sensing of the redox potential is thought to occur in a wide range of diverse signal transduction pathways. Moreover, these redox sensing proteins play roles in mediating cellular responses to oxidative stress (e.g., increased cellular proliferation).

One of the primary enzymes involved in the synthesis of cellular thiols is cysteine synthase, which is widely distributed in human tissues, where it catalyzes the synthesis of cysteine from serine. The absorption of cystine and structurally-related amino acids (e.g., ornithine, arginine, and lysine) are mediated by a complex transporter system. The Xc transporter, as well as other enzymes, participate in these cellular uptake mechanisms. Once transported into the cell, cystine is rapidly reduced to cysteine, in an enzymatic reaction which utilizes reduced glutathione (GSH). In the extracellular environment, the concentrations of cystine are typically substantially higher than cysteine, and whereas the reverse is true in the intracellular environment.

The Applicant of the present invention has previously disclosed the use of disodium 2,2′-dithio-bis ethane sulfonate and other dithioethers to: (i) mitigate nephrotoxicity (see, e.g., U.S. Pat. Nos. 5,789,000; 5,866,169; 5,866,615; 5,866,617; and 5,902,610) and (ii) mitigate neurotoxicity (see, e.g., Published U.S. Patent Application No. 2003/0133994); all of which are incorporated herein by reference in their entirety. However, as previously stated, the novel approach of the present invention is to augment the anti-cancer activity of chemotherapeutic agents against the tumor cells by increasing the oxidative stress and/or by decreasing anti-oxidative capacity therein, in a selective manner.

Ideal properties of an anti-cancer augmentation agent, composition, or regimen include maximizing the anti-cancer activity of chemotherapy as measured by an enhancement or augmentation of the anti-cancer and cytotoxic activity of chemotherapy treatment in the form of reduction in tumor size, delay in the progression of cancer, reduction in metastatic appearance of cancer, and improvement in the survival of treated subjects with cancer; (a) by such treatment, alone and/or (b) while concomitantly, in a selective manner, avoiding deleterious chemotherapeutic agent-induced effects on normal (i.e., non-cancerous) cells and tissues.

If an anti-cancer augmentation agent is capable of increasing the therapeutic index of a chemotherapeutic drug, composition, and/or regimen it may lead to significant benefit to the subject by: (i) increasing tumor response rate, increasing the time to tumor progression, delaying or decreasing the onset of metastatic disease, and increasing overall patient survival; (ii) causing a lack of interference with and an observed quantitative augmentation of the cytotoxic action of anti-cancer activity of the concomitantly administered chemotherapeutic agent; (iii) causing a lack of tumor desensitization or drug resistance to the cytotoxic activity of concomitantly administered chemotherapeutic agent(s); (iv) avoiding increased incidence in medically significant treatment-associated toxicities; and/or (v) allowing safe increases in chemotherapeutic index (i.e., increase dosage of, increased frequency of administration of, or the combination of increased dosage and frequency, and number of treatments with a chemotherapeutic agent or combination of agents without increasing the associated toxicities thereof) by allowing increases in dose, frequency, and/or duration of the primary chemotherapy treatment. Thus, if an anti-cancer augmentation agent is capable of increasing the therapeutic index of a pharmacologically active, but otherwise toxic, chemotherapy drug and/or regimen it may lead to a substantial benefit to the subject by increasing tumor response rate, increasing time to tumor progression, and overall patient survival.

Accordingly, there remains a highly important and, as yet, unmet need for agents, compositions, or regimens which cause the augmentation of the anti-cancer activity of chemotherapeutic agents (i.e., enhancement of the anti-cancer cytotoxic action of chemotherapy agents) and methods of their administration that are optimally capable of acting additively or synergistically with one or more chemotherapeutic agents in reducing, preventing, mitigating, and/or delaying neoplastic disease in subjects in a selective manner.

### SUMMARY OF THE INVENTION

The invention described and claimed herein has many attributes and embodiments including, but not limited to, those set forth or described or referenced in this Summary section. However, it should be noted that this Summary is not intended to be all-inclusive, nor is the invention described and claimed herein limited to, or by, the features or embodiments identified in said Summary. Moreover, this Summary is included for purposes of illustration only, and not restriction.

The present invention includes methods, formulations and devices, and uses of the foregoing. These methods, formulations, and devices function in: (i) the augmentation of the anti-cancer activity of chemotherapy treatment in reducing, preventing, mitigating, delaying the onset of, attenuating the severity of, and/or hastening the resolution of the deleterious physiological manifestations of cancer in a subject who received one or more chemotherapeutic agents, in a selective manner and/or (ii) concomitantly avoiding deleterious chemotherapeutic agent-induced effects on non-cancerous cells and tissues.

Augmentation of anti-cancer activity may cause the enhancement of the cytotoxic action of chemotherapy agents by acting in an additive or synergistic cytotoxic manner with said chemotherapeutic agents in a stimulatory (i.e., inducing oxidative stress) or depletive (i.e., decreasing anti-oxidative capacity) manner within the tumor cells, while concurrently reducing, preventing, mitigating, and/or delaying said deleterious physiological manifestations of said cancer in subjects suffering therefrom, wherein the enhancement of the cytotoxic action of chemotherapy agents occurs in a selective manner, which avoids deleterious chemotherapeutic agent-induced effects on normal (i.e., non-cancerous) cells and tissues.

Similarly, an anti-cancer augmentation agent is a compound, formulation, or agent which is capable of eliciting the augmentation of the anti-cancer cytotoxic action of chemotherapeutic agents, alone, and may further provide benefit of reducing, preventing, mitigating, and/or delaying the deleterious physiological manifestations of cancer in subjects suffering therewith.

Methods include administering to a subject who has received, is currently receiving, or will receive one or more chemotherapeutic agents, an effective amount of the dithio-containing compounds of the present invention, which include 2,2′-dithio-bis-ethane sulfonate, a pharmaceutically-acceptable salt thereof, an analog thereof, and the compounds of Formula (I), administered to said subject, by way of non-limiting example, at a rate of about 0.1 g/min. to about 2.0 g/min. in order to elicit anti-cancer augmentation of said chemotherapy treatment.

In one embodiment, an effective amount of the dithio-containing compounds of the present invention, which include 2,2′-dithio-bis-ethane sulfonate, a pharmaceutically-acceptable salt thereof, an analog thereof, and the compounds of Formula (I), is administered to a subject who received one or more chemotherapeutic agents, wherein said dithio-containing compound is administered to said subject at a rate of about 0.2 g/min. to about 1.0 g/min. in order to elicit anti-cancer augmentation of said co-existing or concurrent or contemporaneously administered chemotherapy treatment.

In another embodiment, an effective amount of the dithio-containing compounds of the present invention, which include 2,2′-dithio-bis-ethane sulfonate, a pharmaceutically-acceptable salt thereof, an analog thereof, and the compounds of Formula (I), is administered to a subject who received one or more chemotherapeutic agents, wherein said dithio-containing compound is administered to said subject at a rate of about 0.7 g/min. in order to elicit anti-cancer augmentation of said chemotherapy treatment.

In yet another embodiment, an effective amount of the dithio-containing compounds of the present invention, which include 2,2′-dithio-bis-ethane sulfonate, a pharmaceutically-acceptable salt thereof, an analog thereof, and the compounds of Formula (I), is administered to a subject who received one or more chemotherapeutic agents, over a period of about 45 minutes in order to elicit anti-cancer augmentation of said chemotherapy treatment.

In one embodiment, the total dose of the dithio-containing compounds of the present invention, which include 2,2′-dithio-bis-ethane sulfonate, a pharmaceutically-acceptable salt thereof, an analog thereof, and the compounds of Formula (I), is administered to a subject who received one or more chemotherapeutic agents, wherein the total dose of said dithio-containing compound administered to said subject in need thereof is from about 2.0 g/m² to about 60 g/m² in order to elicit anti-cancer augmentation of said chemotherapy treatment. One preferred dose of said dithio-containing compounds is about 18.4 g/m². Particularly preferred, is the administration of one or more doses of said dithio-containing compounds of the present invention to said subject over about 45 minutes.

The present invention also discloses and claims methods of augmenting the anti-cancer activity of chemotherapeutic agent(s) administered to a subject who received one or more chemotherapeutic agents, wherein said method comprises administering to said subject in need thereof an effective amount of the dithio-containing compounds of the present invention, which include 2,2′-dithio-bis-ethane sulfonate, a pharmaceutically-acceptable salt thereof, an analog thereof, and the compounds of Formula (I), at a rate of about 0.1 g/min. to about 4.6 g/min., at a total dose of about 4 g/m² to about 41 g/m² in order to elicit anti-cancer augmentation of said chemotherapy treatment. Preferred is administration of a total dose of about 18.4 g/m² at a rate of about 0.1 g/min. to about 4.6 g/min. of said dithio-containing compound to said subject. Particularly preferred is administration of a total dose of about 18.4 g/m² over about 45 minutes of said dithio-containing compound to said subject at a rate of about 0.4 g/m²/min.

In another embodiment, an effective amount of the dithio-containing compounds of the present invention, which include 2,2′-dithio-bis-ethane sulfonate, a pharmaceutically-acceptable salt thereof, an analog thereof, and the compounds of Formula (I), is administered to a subject who received one or more chemotherapeutic agents, wherein said dithio-containing compound is administered to a subject at a rate of about 1 mg/mL/min. to about 50 mg/mL/min. in order to elicit anti-cancer augmentation of said chemotherapy treatment.

In another embodiment of the invention, an effective amount of the dithio-containing compounds of the present invention, which include 2,2′-dithio-bis-ethane sulfonate, a pharmaceutically-acceptable salt thereof, an analog thereof, and the compounds of Formula (I), is administered to a subject who received one or more chemotherapeutic agents, wherein said dithio-containing compound is administered to said subject at a rate of about 7 mg/mL/min. in order to elicit anti-cancer augmentation of said chemotherapy treatment. In one embodiment the dithio-containing compound of the present invention is administered to said subject in need thereof over a period of about 45 minutes in order to elicit anti-cancer augmentation of said chemotherapy treatment. In another embodiment the dithio-containing compound is administered to said subject who has received, is currently receiving, or will receive one or more chemotherapeutic agents, wherein a formulation having a concentration of about 100 mg/mL of a dithio-containing compound is administered in sufficient quantity to said subject in order to elicit anti-cancer augmentation of said chemotherapy treatment. In yet another embodiment, the dithio-containing compound is administered alone to said subject over a period of about 45 minutes and in a formulation having a concentration of about 100 mg/mL of said dithio-containing compound.

The present invention also discloses and claims methods of augmenting the anti-cancer activity of chemotherapeutic agent(s) administered to a subject who received one or more chemotherapeutic agents, wherein said method comprises administering to said subject in need thereof, an effective amount of the dithio-containing compounds of the present invention, which include 2,2′-dithio-bis-ethane sulfonate, a pharmaceutically-acceptable salt thereof, an analog thereof, and the compounds of Formula (I), wherein said composition has an osmolarity that is about 0.1- to about 5-times the osmolarity of the normal range of plasma osmolarity in order to elicit anti-cancer augmentation of said chemotherapy treatment. In another aspect of the present invention, said composition has an osmolarity that is about 2- to about 4-times the normal range of plasma osmolarity. In yet another aspect of the invention, said composition has an osmolarity that is about 3-times the normal range of plasma osmolarity.

It should be noted that any one of the aforementioned variables of dose of the dithio-containing compounds of the present invention; rate of administration; concentration; formulation; osmolarity; and infusion time may be combined with any one or more other of these variables, in the amounts and/or ranges set forth, to create a composition or formulation or method of administration for one or more of the described anti-cancer augmentation agents.

In one embodiment, the dithio-containing compound of the present invention is a disodium salt.

In other embodiments, the dithio-containing compound of the present invention is a pharmaceutically-acceptable salt, which include but are not limited to: (i) a monosodium salt; (ii) a sodium potassium salt; (iii) a dipotassium salt; (iv) a calcium salt; (v) a magnesium salt; (vi) a manganese salt; (vii) an ammonium salt; and (viii) a monopotassium salt. It should be noted that mono- and di-potassium salts are only administered to a subject if the total dose of potassium administered at any given point in time is not greater than 100 Meq., the subject is not hyperkalemic, and/or the subject does not have a condition that would predispose the subject to hyperkalemia (e.g., renal failure).

Embodiments of the present invention also include controlled or other doses, dosage forms, formulations, compositions and/or devices containing one or more chemotherapeutic agents and a dithio-containing compound of the present invention, which include 2,2′-dithio-bis-ethane sulfonate, a pharmaceutically-acceptable salt, an analog thereof, and the compounds of Formula (I), including: doses and dosage forms for (i) oral (e.g., tablet, suspension, solution, gelatin capsule (hard or soft), sublingual, dissolvable tablet, troche, and the like); (ii) injection (e.g., subcutaneous administration, intradermal administration, subdermal administration, intramuscular administration, depot administration, intravenous administration, intra-arterial administration, and the like); (iii) intra-cavitary (e.g., into the intrapleural, intraperitoneal, intravesicular, and/or intrathecal spaces); (iv) per rectum (e.g., suppository, retention enema); and (v) topical administration routes.

In yet another embodiment, a composition comprising one or more chemotherapeutic agents and a dithio-containing compound of the present invention, which include, 2,2′-dithio-bis-ethane sulfonate, a pharmaceutically-acceptable salt thereof, an analog thereof, and the compounds of Formula (I), is administered using the rates and/or times described herein, with or without using the concentrations and/or osmolarity ranges described herein, alone or in conjunction with a dose as described herein.

In another embodiment, a composition comprising one or more chemotherapeutic agents and a dithio-containing compound of the present invention, which include, 2,2′-dithio-bis-ethane sulfonate, a pharmaceutically-acceptable salt thereof, an analog thereof, and the compounds of Formula (I), is administered from about once a day to about once every five weeks, including about once a week or less, about once every two weeks or less, about once every three weeks or less, about once every four weeks or less, about once every five weeks or less, and any daily or weekly interval in between.

In one embodiment, a composition comprising one or more chemotherapeutic agents and a dithio-containing compound of the present invention, which include, 2,2′-dithio-bis-ethane sulfonate, a pharmaceutically-acceptable salt thereof, an analog thereof, and the compounds of Formula (I), is utilized to elicit anti-cancer augmentation of said chemotherapy treatment.

In one embodiment, a dithio-containing compound of the present invention, which include, 2,2′-dithio-bis-ethane sulfonate, a pharmaceutically-acceptable salt thereof, an analog thereof, and the compounds of Formula (I), is administered with a chemotherapeutic agent, either a single chemotherapeutic agent or multiple chemotherapeutic agent combinations, without limitation, in accordance with medical indications involving the proper treatment of a subject's cancer(s). In various embodiments of the present invention, the chemotherapeutic agent is, by way of non-limiting example, one or more of the following compounds: a fluropyrimidine; a pyrimidine nucleoside; a purine nucleoside; an antifolate, a platinum analog; an anthracycline/anthracenedione; an epipodophyllotoxin; a camptothecin; a hormone, a hormonal analog; an antihormonal; an enzyme, protein, peptide, or polyclonal and monoclonal antibody; a vinca alkaloid; a taxane; an epothilone; an antimicrotubule agent; an alkylating agent; an antimetabolite; a topoisomerase inhibitor; an antiviral; or another cytotoxic and/or cytostatic agent.

In another embodiment, the method comprises one or more hydration step(s). Hydration comprises the administration of various fluids to the subject in need thereof for purposes of facilitating medical treatment to said subject. Such hydration may serve, e.g., to replace or increase internal fluid levels.

In yet another embodiment, the method comprises administering one or more pre-therapy medication(s). Pre-medications include, for example, antihistamines, steroids, antimetics, and 5-HT3 antagonists. Pre-therapy may be administered according to methods known within the art and in accordance with the patient's medical condition.

In one embodiment, the method is carried out to treat one or more cancers in a subject. In another embodiment, the subject is a human. Said cancer or cancers may be human cancers, which may include, for example, one or more cancers of the: ovary, breast, lung, esophagus, bladder, stomach, pancreas, liver (e.g., bile ducts, gall bladder, and Ampulla of Vater), testes, germ cell, bone, cartilage, head, neck, oral mucosa, colorectal area, anus, kidney, uroepithelium, central nervous system, prostate, endometrium, cervix, uterus, fallopian tube, peripheral nervous system, and various other cancers including melanoma, mesothelioma, myeloma, lymphoma, leukemia, and Kaposi's sarcoma.

### DETAILED DESCRIPTION OF THE INVENTION

The descriptions and embodiments set forth herein are not intended to be exhaustive, nor do they limit the present invention to the precise forms disclosed. They are included to illustrate the principles of the invention, and its application and practical use by those skilled in the art.

### Definitions

“Scaffold” or “generic structural formula” means the fixed structural part of the molecule of the formula given.

“Nucleophile” means an ion or molecule that donates a pair of electrons to an atomic nucleus to form a covalent bond; the nucleus that accepts the electrons is called an electrophile. This occurs, for example, in the formation of acids and bases according to the Lewis concept, as well as in covalent carbon bonding in organic compounds.

“Fragments”, “Moieties” or “Substituent Groups” are the variable parts of the molecule, designated in the formula by variable symbols, such as Rₓ, X or other symbols. Substituent Groups may consist of one or more of the following:

“Cₓ-C_{y} alkyl” generally means a straight or branched-chain aliphatic hydrocarbon containing as few as x and as many as y carbon atoms. Examples include “C₁-C₆ alkyl” (also referred to as “lower alkyl”), which includes a straight or branched chain hydrocarbon with no more than 6 total carbon atoms, and C₁-C₁₆ alkyl, which includes a hydrocarbon with as few as one up to as many as sixteen total carbon atoms, and the like. In the present application, the term “alkyl” is defined as comprising a straight or branched chain hydrocarbon of between 1 and 20 atoms, which can be saturated or unsaturated, and may include heteroatoms such as nitrogen, sulfur, and oxygen;

“Cₓ-C_{y} alkylene” means a bridging moiety formed of as few as “x” and as many as “y” —CH₂— groups. In the present invention, the term “alkylene” or “lower alkylene” is defined as comprising a bridging hydrocarbon having from 1 to 6 total carbon atoms which is bonded at its terminal carbons to two other atoms (—CH₂—)ₓ where x is 1 to 6;

“Cₓ-C_{y} alkenyl or alkynyl” means a straight or branched chain hydrocarbon with at least one double bond(alkenyl) or triple bond (alkynyl) between two of the carbon atoms;

“Halogen” or “Halo” means chloro, fluoro, bromo or iodo;

“Acyl” means —C(O)—R, where R is hydrogen, Cₓ-C_{y} alkyl, aryl, Cₓ-C_{y} alkenyl, Cₓ-C_{y} alkynyl, and the like;

“Acyloxy” means —O—C(O)—R, where R is hydrogen, Cₓ-C_{y}alkyl, aryl, and the like;

“Cₓ-C_{y} Cycloalkyl” means a hydrocarbon ring or ring system consisting of one or more rings, fused or unfused, wherein at least one of the ring bonds is completely saturated, with the ring(s) having from x to y total carbon atoms;

“Aryl” generally means an aromatic ring or ring system consisting of one or more rings, preferably one to three rings, fused or unfused, with the ring atoms consisting entirely of carbon atoms. In the present invention, the term “aryl” is defined as comprising an aromatic ring system, either fused or unfused, preferably from one to three total rings, with the ring elements consisting entirely of 5-8 carbon atoms;

“Arylalkyl” means an aryl moiety as defined above, bonded to the scaffold through an alkyl moiety (the attachment chain);

“Arylalkenyl” and “Arylalkynyl” mean the same as “Arylalkyl”, but including one or more double or triple bonds in the attachment chain;

“Amine” means a class of organic complexes of nitrogen that may be considered as derived from ammonia (NH₃) by replacing one or more of the hydrogen atoms with alkyl groups. The amine is primary, secondary or tertiary, depending upon whether one, two or three of the hydrogen atoms are replaced. A “short chain anime” is one in which the alkyl group contains from 1 to 10 carbon atoms;

“Ammine” means a coordination analog formed by the union of ammonia with a metallic substance in such a way that the nitrogen atoms are linked directly to the metal. It should be noted the difference from amines, in which the nitrogen is attached directly to the carbon atom;

“Azide” means any group of complexes having the characteristic formula R(N₃)ₓ. R may be almost any metal atom, a hydrogen atom, a halogen atom, the ammonium radical, a complex [CO(NH₃)₆], [Hg(CN)₂M], (with M=Cu, Zn, Co, Ni) an organic radical like methyl, phenyl, nitrophenol, dinitrophenol, p-nitrobenzyl, ethyl nitrate, and the like. The azide group possesses a chain structure rather than a ring structure;

“Imine” means a class of nitrogen-containing complexes possessing a carbon-to-nitrogen double bond (i.e., R—CH═NH);

“Heterocycle” means a cyclic moiety of one or more rings, preferably one to three rings, fused or unfused, wherein at least one atom of one of the rings is a non-carbon atom. Preferred heteroatoms include oxygen, nitrogen and sulfur, or any combination of two or more of those atoms. The term “Heterocycle” includes furanyl, pyranyl, thionyl, pyrrolyl, pyrrolidinyl, prolinyl, pyridinyl, pyrazolyl, imidazolyl, triazolyl, tetrazolyl, oxathiazolyl, dithiolyl, oxazolyl, isoxazolyl, oxadiazolyl, pyridazinyl, pyrimidinyl, pyrazinyl, piperazinyl, oxazinyl, thiazolyl, and the like; and

“Substituted” modifies the identified fragments (moieties) by replacing any, some or all of the hydrogen atoms with a moiety (moieties) as identified in the specification. Substitutions for hydrogen atoms to form substituted complexes include halo, alkyl, nitro, amino (also N-substituted, and N,N di-substituted amino), sulfonyl, hydroxy, alkoxy, phenyl, phenoxy, benzyl, benzoxy, benzoyl, and trifluoromethyl.

As utilized herein, the definitions for the terms “Adverse Event” (effect or experience), “Adverse Reaction”, and unexpected adverse reaction have previously been agreed to by consensus of the more than 30 Collaborating Centers of the WHO International Drug Monitoring Centre (Uppsala, Sweden). See, Edwards, I. R., et al., Harmonisation in Pharmacovigilance Drug Safety 10(2):93-102 (1994). The following definitions, with input from the WHO Collaborative Centre, have been agreed to:

1. Adverse Event (Adverse Effect or Adverse Experience)—Any untoward medical occurrence in a patient or clinical investigation subject administered a pharmaceutical product and which does not necessarily have to have a causal relationship with this treatment. An Adverse Event (AE) can therefore be any unfavorable and unintended sign (including an abnormal laboratory finding, for example), symptom, or disease temporally associated with the use of a medicinal product, whether or not considered related to the medicinal product.

2. Adverse Drug Reaction (ADR)—In the pre-approval clinical experience with a new medicinal product or its new usages, particularly as the therapeutic dose(s) may not be established: all noxious and unintended responses to a medicinal product related to any dose should be considered adverse drug reactions. Drug-related Adverse Events are rated from grade 1 to grade 5 and relate to the severity or intensity of the event. Grade 1 is mild, grade 2 is moderate, grade 3 is severe, grade 4 is life threatening, and grade 5 results in the subject's death.

3. Unexpected Adverse Drug Reaction—An adverse reaction, the nature or severity of which is not consistent with the applicable product information.

Serious Adverse Event or Adverse Drug Reaction: A Serious Adverse Event (experience or reaction) is any untoward medical occurrence that at any dose:
(1) Results in death or is life-threatening. It should be noted that the term “life-threatening” in the definition of “serious” refers to an event in which the patient was at risk of death at the time of the event; it does not refer to an event which hypothetically might have caused death if it were more severe.

### (2) Requires inpatient hospitalization or prolongation of existing hospitalization.

### (3) Results in persistent or significant disability/incapacity, or

### (4) Is a congenital anomaly/birth defect.

As utilized herein the term “cancer” refers to all known forms of cancer including, solid forms of cancer (e.g., tumors), lymphomas, and leukemias.

As used herein, the term “anti-cancer augmentation” and “augmentation of anti-cancer activity” is defined herein as producing one or more of the following physiological effects: (i) the enhancement of the cytotoxic activity of chemotherapy agents by acting in an additive or synergistic cytotoxic manner with said chemotherapeutic agents in a stimulatory (i.e., inducing oxidative stress) or depletive (i.e., decreasing anti-oxidative capacity) manner within the tumor cells; (ii) reducing, preventing, mitigating, and/or delaying said deleterious physiological manifestations of said cancer in subjects suffering therewith; (iii) selectively sensitizing cancer cells to the anti-cancer activity of chemotherapeutic agents; and/or (iv) restoring apoptotic effects or sensitivity in tumor cells.

As used herein, the term “anti-cancer augmentation agent” is defined herein as a compound, formulation, or agent which is capable of eliciting one or more of the following physiological effects: (i) the enhancement of the cytotoxic activity of chemotherapeutic agents by acting in a synergistic manner with said chemotherapeutic agents in a stimulatory (i.e., inducing oxidative stress) or depletive (i.e., decreasing anti-oxidative capacity) manner within the tumor cells in subjects suffering therefrom and/or (ii) the enhancement of the cytotoxic activity of chemotherapeutic agents is in a selective manner, which causes the reduction, mitigation, prevention, or delay of deleterious chemotherapeutic agent-induced effects on normal (i.e., non-cancerous) cells and tissues.

As used herein “chemotherapeutic agent” or “chemotherapy agent” or “antineoplastic agent” refer to an agent that reduces, prevents, mitigates, limits, and/or delays the growth of metastases or neoplasms, or kills neoplastic cells directly by necrosis or apoptosis of neoplasms or any other mechanism, or that can be otherwise used, in a pharmaceutically-effective amount, to reduce, prevent, mitigate, limit, and/or delay the growth of metastases or neoplasms in a subject with neoplastic disease. Chemotherapeutic agents include, for example, fluropyrimidines; pyrimidine nucleosides; purine nucleosides; anti-folates, platinum complexes; anthracyclines/anthracenediones; epipodophyllotoxins; camptothecins; hormones; hormonal complexes; antihormonals; enzymes, proteins, peptides and antibodies; vinca alkaloids; taxanes; epothilones; antimicrotubule agents; alkylating agents; antimetabolites; topoisomerase inhibitors; antivirals; and miscellaneous cytotoxic and cytostatic agents.

As utilized herein, the term “chemotherapy” or “chemotherapeutic regimen(s)” refers to treatment using the above-mentioned chemotherapeutic agents with or without the dithio-containing compound of the present invention.

As used herein, the terms “a dithio-containing compound of the present invention”, “dithio-containing compounds of the present invention”, or “dithio-containing compound(s)” includes all molecules, unless specifically identified otherwise, that share substantial structural and/or functional characteristics with the 2,2′-dithio-bis-ethane sulfonate parent compound and include the compounds of Formula (I) which refers to compounds possessing the generic structural formula:

X—S—S—R₁—R₂:

wherein;

R₁ is a lower alkylene, wherein R₁ is optionally substituted by a member of the group comprising: aryl, hydroxy, alkoxy, aryloxy, mercapto, alkylthio or arylthio, for a corresponding hydrogen atom;

R₂ is sulfonate or phosphonate;

X is a sulfur-containing amino acid or a peptide comprising from 2-10 amino acids; wherein X is optionally substituted by a member of the group comprising: lower alkyl, lower alkenyl, lower alkynyl, aryl, alkoxy, aryloxy, mercapto, alkylthio or hydroxy for a corresponding hydrogen atom.

The compounds of Formula (I) include pharmaceutically-acceptable salts thereof, as well as prodrugs, analogs, conjugates, hydrates, solvates and polymorphs, as well as stereoisomers (including diastereoisomers and enantiomers) and tautomers thereof. Also included, is the key metabolite of disodium 2,2′-dithio-bis-ethane sulfonate, 2-mercapto ethane sulfonate sodium (also known in the literature as mesna). Various compounds of Formula (I), and their synthesis are described in published U.S. Patent Application No. 2005/0256055, the disclosure of which is hereby incorporated by reference in its entirety.

As used herein, an “effective amount” or a “pharmaceutically-effective amount” in reference to the compounds or compositions of the instant invention refers to the dosage that is sufficient to induce a desired biological, pharmacological, or therapeutic outcome in a subject with neoplastic disease. That result can be: (i) cure or remission of previously observed cancer(s); (ii) shrinkage of tumor size; (iii) reduction in the number of tumors; (iv) delay or prevention in the growth or reappearance of cancer; (v) selectively sensitizing cancer cells to the anti-cancer activity of chemotherapeutic agents; (vi) restoring apoptotic effects or sensitivity in tumor cells; and/or (vii) increasing the survival of the patient, alone or while concurrently experiencing reduction, prevention, mitigation, delay, shortening the time to resolution of, alleviation of the signs or symptoms of the incidence or occurrence of an expected side-effect(s), toxicity, disorder or condition, or any other untoward alteration in the patient.

As used herein the term “g/m²” represents the amount of a given compound or formulation in grams per square meter of the total body surface area of the subject to whom the compound or formulation is administered.

“Osmolarity” is a measure of the osmoles of solute per kilogram of solvent. For purposes of calculating osmolarity, salts are presumed to dissociate into their component ions. For example, a mole of glucose in solution is one osmole, whereas a mole of sodium chloride in solution is two osmoles (one mole of sodium and one mole of chloride). Both sodium and chloride ions affect the osmotic pressure of the solution. The equation to determine the osmolarity of a solution is given by Osm=ΦnC, where Φ is the osmotic coefficient and accounts for the degree of dissociation of the solute; Φ is between 0 and 1, where 1 indicates 100% dissociation; n is the number of particles into which a molecule dissociates (for example: Glucose equals 1 and NaCl equals 2); and C is the molar concentration of the solution.

As used herein, the term “pre-treatment” comprises the administration of one or more medications, said administration occurring at least one day prior to chemotherapy, prior to each chemotherapy treatment, immediately prior to each chemotherapy treatment, concomitantly with or simultaneously during chemotherapy treatment, immediately subsequent to chemotherapy, subsequent to chemotherapy, any combination of the foregoing, and/or according to methods known within the art and in accordance with the patient's medical condition.

“Pharmaceutically-acceptable salt” means salt derivatives of drugs which are accepted as safe for human administration. In the present invention, the dithio-containing compound of the present invention includes pharmaceutically-acceptable salts, which include but are not limited to: (i) a monosodium salt; (ii) a disodium salt; (iii) a sodium potassium salt; (iv) a dipotassium salt; (v) a calcium salt; (vi) a magnesium salt; (vii) a manganese salt; (viii) an ammonium salt; and (ix) a monopotassium salt.

As used herein the terms “reactive oxygen species (ROS)” and “reactive nitrogen species (RNS)” refer to ionic species which may result from a variety of metabolic and/or environmental processes. By way of non-limiting example, intracellular ROS (e.g., hydrogen peroxide: H₂O₂, superoxide anion: O₂⁻, hydroxyl radical: OH⁻, nitric oxide, and the like) may be generated by several mechanisms: (i) by the activity of radiation; (ii) during xenobiotic and drug metabolism; and (iii) under relative hypoxic, ischemic and catabolic metabolic conditions.

As used herein, the term “receive” or “received” refers to a subject who has cancer and who has received, is currently receiving, or will receive one or more chemotherapeutic agents and/or dithio-containing compounds of the present invention.

As used herein the term “redox state”, “redox potential”, or “oxidative/reductive state” of any particular biological environment can be defined as the sum of oxidative and reductive processes occurring within that environment, which affects the extent to which molecules are oxidized or reduced within it. The redox potential of biological ions or molecules is a measure of their tendency to lose an electron (i.e., thereby becoming oxidized). Under normal physiological circumstances, most intracellular biological systems are predominantly found in a reduced state. Within cells, thiols (R—SH) such as glutathione (GSH) are maintained in their reduced state, as are the nicotinamide nucleotide coenzymes NADH and NADPH. Conversely, plasma is generally an oxidizing environment due to the high partial pressure of oxygen and the relative absence of disulfide reducing enzymes. Physiological circumstances can, however, arise which alter the overall redox balance and lead to a more oxidizing environment on cells. In biological systems, this activity arises as a result of oxidative stress and physiological systems have evolved to preserve, protect, and control the normal reducing environment. However, when oxidative stress overwhelms these protective mechanisms, oxidative damage and profound biological changes can occur. Cancer cells have been observed to have the ability to mount more effective anti-oxidative responses to oxidative stress (in comparison to normal, i.e., non-cancerous, cells), thereby leading to a survival advantage and the ability to resist or escape the anti-cancer and cytotoxic action of chemotherapeutic agent(s).

As used herein the term “synergism” or “synergistic” means the anti-cancer activity achieved by the above-defined “dithio-containing compounds” in combination with chemotherapeutic agent(s) is greater than the anti-cancer activity achieved by either form of treatment individually. For example, this may be mathematically expressed as the synergistic result of treatment with Drugs A+B administered together (as taught herein)=Result C>Drug A Result, alone+Drug B Result, alone. In contrast, a purely additive result may be mathematically expressed as: Drugs A+B administered together=Result C=Drug A Result, alone+Drug B Result, alone. In the foregoing examples, Drug A can represent Formula (I) compounds and the observed treatment result alone or combined, and Drug B can represent any single chemotherapy agent or combination of chemotherapy agents that are administered alone.

The term “solvate” or “solvates” refers to a molecular complex of a compound such as a dithio-containing compound of the present invention with one or more solvent molecules. Such solvent molecules are those commonly used in the pharmaceutical art (e.g., water, ethanol, and the like). The term “hydrate” refers to the complex where the solvent molecule is water.

As used herein, the term “reducing” includes preventing, attenuating the overall severity of, delaying the initial onset of, and/or expediting the resolution of the acute and/or chronic pathophysiology associated with malignancy in a subject by the augmentation of the cytotoxic/anti-cancer activity of chemotherapy agents by acting in an additive or synergistic manner with said chemotherapeutic agents; and/or in a selective manner; and/or while avoiding deleterious chemotherapeutic agent-induced effects on normal (i.e., non-cancerous) cells and tissues.

As used herein, “treatment schedule time” means the difference in schedule of administration time, including: (i) the amount of drug administered per day or week; (ii) the amount of drug administered per day or week per m² of body surface area; and (iii) the amount of drug administered per day or week per kg of body weight.

As used herein, “difference in administration of drug treatment time”, means permitting administration of treatment to occur in materially less time (a reduction in time from, e.g., 4 hours to 1 hour, from one day to 6 hours, and the like) thereby allowing the patient to minimize time in the outpatient or hospitalized treatment time.

### I. Activity of 2,2′-Dithio-Bis Ethane Sulfonate on Physiological Cellular Thiols and Non-Protein Sulfhydryls (NPSH)

As the number of agents and treatments for cancer, as well as the number of subjects receiving one or more of these chemotherapeutic agents concomitantly, has increased, clinicians and researchers are fervently seeking to fully elucidate the biological, chemical pharmacological, and cellular mechanisms which are responsible for the pathogenesis and pathophysiology of the various adverse disease manifestations, as well as how these chemotherapeutic drugs exert their anti-cancer and cytotoxic activity on a biochemical and pharmacological basis. Unfortunately, as previously discussed, there is no treatment presently available which is generally safe and effective for augmenting the anti-cancer activity of chemotherapeutic agents for either preventing or delaying the initial onset of, attenuating the overall severity of, and/or expediting the resolution of the acute or chronic pathophysiology associated with malignancy in subjects suffering therefrom, wherein the enhancement of the cytotoxic activity of chemotherapeutic agent or agents is in a selective manner, which attenuates or prevents deleterious chemotherapeutic agent-induced effects on normal (i.e., non-cancerous) cells and tissues. The potential pathophysiological mechanisms responsible for such these aforementioned manifestations are not fully known, and in many cases are the topic of energetic debate. Furthermore, as described herein, with the exception of the novel conception and practice of this invention, there are no agents currently approved that are associated with enhancement of tumor cell kill or augmented cytotoxicity on cancer cells in a selective manner while avoiding deleterious chemotherapeutic agent-induced effects on normal (i.e., non-cancerous) cells and tissues.

The mechanisms by which the dithio-containing compounds of the present invention (which include 2,2′-dithio-bis-ethane sulfonate, a pharmaceutically-acceptable salt thereof, an analog thereof, and the compounds of Formula (I)) function in the augmentation of the anti-cancer activity of chemotherapeutic agent(s) involves several novel pharmacological and physiological factors, including but not limited to:
(i) a prevention, compromise and/or reduction in the normal increase, responsiveness, or in the concentration and metabolism of glutathione/cysteine and other physiological cellular thiols; these antioxidants and enzymes are increased in concentration and/or activity, respectively, in response to the induction of intracellular oxidative stress which may be caused by exposure to chemotherapeutic agents in tumor cells. The dithio-containing compounds of the present invention exert an oxidative activity by the intrinsic composition of the molecule itself (i.e., an oxidized disulfide), as well as by oxidizing free thiols to form oxidized disulfides (i.e., by non-enzymatic SN2-mediated reactions, wherein attack of a thiol/thiolate upon a disulfide leads to the departure of the more acidic thiol group. As the thiolate group is far more nucleophilic than the corresponding thiol, the attack is believed to be via the thiolate), and by the pharmacological depletion and metabolism of reductive physiological free thiols (e.g., glutathione, cysteine, and homocysteine). These pharmacological activities will thus have an augmenting effect on cytotoxic chemotherapy administration to patients with cancer, and additional anti-cancer activity will result from the administration of a dithio-containing compound of the present invention, augmenting the drug efficacy, and reducing the tumor-mediated resistance of the various co-administered chemotherapeutic agents, e.g., platinum- and alkylating agent-based drug efficacy and tumor-mediated drug resistance;
(ii) thioredoxin inactivation by a dithio-containing compound of the present invention, thereby increasing apoptotic sensitivity and decreasing mitogenic/cellular replication signaling in cancer cells;
(iii) a key metabolite of disodium 2,2′-dithio-bis-ethane sulfonate, which metabolite is known as 2-mercapto ethane sulfonate sodium (i.e., also known in the literature as mesna) possesses intrinsic cytotoxic activity (i.e., causes apoptosis) in some tumors by an, as yet, unknown mechanism which can kill cancer cells directly; and
(iv) it is believed that dithio-containing compounds of the present invention (and possibly mesna) act by enhancing oxidative stress or compromising the anti-oxidative response of cancerous tumor cells, and may enhance their oxidative biological and physiological state and thereby increase the amount of oxidative damage (e.g., mediated by ROS, RNS or other mechanisms) in tumor cells exposed to chemotherapy, thereby enhancing cytotoxicity/apoptosis of chemotherapy agents. Thus, by enhancing oxidative stress and/or reducing or compromising the total anti-oxidative capacity or responsiveness of cancer tumor cells, a marked increase in anti-cancer activity can be achieved. It is believed by the Applicant of the present invention that this is a key anti-cancer augmentation mechanism of action that may act in concert with other mechanisms of anti-cancer augmentation of dithio-containing compounds of the present invention with very important implications for treatment.

Compositions and formulations comprising a dithio-containing compound of the present invention may either be given: (i) in a stimulatory (i.e., inducing oxidative stress) or depletive (i.e., decreasing anti-oxidative capacity) manner to a cancer patient prior to the administration of an oxidative stress-inducing chemotherapeutic agent or agents in order to sensitize the neoplasm so as to augment the tumor cytotoxicity of chemotherapy, while at the same time the same compositions and formulations prevent or mitigate the development of chemotherapy-induced side-effects in normal tissues; (ii) in a therapeutic manner, as a cancer patient begins a chemotherapy cycle, in order to augment the activity of the oxidative stress induced by the chemotherapeutic agent or agents; and/or (iii) in a subsequent manner (i.e., after said chemotherapy cycle) in order to continue the induction or maintenance of the oxidative stress process in cancer cells and to prevent or mitigate any chemotherapy-associated side-effect(s). Additionally, the aforementioned compositions and formulations may be given in an identical manner to augment the anti-cancer activity of a cytotoxic agent by any clinically-beneficial mechanism(s).

### Glutathione and Cysteine

Glutathione (GSH), a tripeptide (γ-glutamyl-cysteinyl-glycine) serves a highly important role in both intracellular and extracellular redox balance. It is the main derivative of cysteine, and the most abundant intracellular non-protein thiol, with an intracellular concentration approximately 10-times higher than other intracellular thiols. Within the intracellular environment, glutathione is maintained in the reduced form (GSH) by the action of glutathione reductase and NADPH. Under conditions of oxidative stress, however, the concentration of GSH becomes markedly depleted. Glutathione functions in many diverse roles including, but not limited to, regulating antioxidant defenses, detoxification of drugs and xenobiotics, and in the redox regulation of signal transduction. As an antioxidant, glutathione may serve to scavenge intracellular free radicals directly, or act as a co-factor for various other protection enzymes. In addition, glutathione may also have roles in the regulation of immune response, control of cellular proliferation, and prostaglandin metabolism. Glutathione is also particularly relevant to oncology treatment because of its recognized roles in tumor-mediated drug resistance to chemotherapeutic agents and ionizing radiation. Glutathione is able to conjugate electrophilic drugs such as alkylating agents and cisplatin under the action of glutathione S-transferases. Recently, GSH has also been linked to the efflux of other classes of agents such as anthracyclines via the action of the multidrug resistance-associated protein (MRP). In addition to drug detoxification, GSH enhances cell survival by functioning in antioxidant pathways that reduce reactive oxygen species, and maintain cellular thiols (also known as non-protein sulfhydryls (NPSH)) in their reduced states. See, e.g., Kigawa J, et al., Gamma-glutamyl cysteine synthetase up-regulates glutathione and multidrug resistance-associated protein in patients with chemoresistant epithelial ovarian cancer. Clin. Cancer Res. 4:1737-1741(1998).

Cysteine, another important NPSH, as well as glutathione are also able to prevent DNA damage by radicals produced by ionizing radiation or chemical agents. Cysteine concentrations are typically much lower than GSH when cells are grown in tissue culture, and the role of cysteine as an in vivo cytoprotector is less well-characterized. However, on a molar basis cysteine has been found to exhibit greater protective activity on DNA from the side-effect(s) of radiation or chemical agents. Furthermore, there is evidence that cysteine concentrations in tumor tissues can be significantly greater than those typically found in tissue culture.

A number of studies have examined GSH levels in a variety of solid human tumors, often linking these to clinical outcome See, e.g., Hochwald, S. N., et al., Elevation of glutathione and related enzyme activities in high-grade and metastatic extremity soft tissue sarcoma. American Surg. Oncol. 4:303-309 (1997); Ghazal-Aswad, S., et al., The relationship between tumour glutathione concentration, glutathione S-transferase isoenzyme expression and response to single agent carboplatin in epithelial ovarian cancer patients. Br. J. Cancer 74:468-473 (1996); Berger, S. J., et al., Sensitive enzymatic cycling assay for glutathione: Measurement of glutathione content and its modulation by buthionine sulfoximine in vivo and in vitro human colon cancer. Cancer Res. 54:4077-4083 (1994). Wide ranges of tumor GSH concentrations have been reported, and in general these have been greater (i.e., up to 10-fold) in tumors compared to adjacent normal tissues. Most researchers have assessed the GSH content of bulk tumor tissue using enzymatic assays, or GSH plus cysteine using HPLC.

In addition, cellular thiols/non-protein sulfhydryls (NPSH), e.g., glutathione, have been associated with increased tumor resistance to therapy by mechanisms that include, but are not limited to: (i) conjugation and excretion of chemotherapeutic agents; (ii) direct and indirect scavenging of reactive oxygen species (ROS) and reactive nitrogen species (RNS); and (iii) maintenance of the “normal” intracellular redox state. Low levels of intracellular oxygen within tumor cells (i.e., tumor hypoxia) caused by aberrant structure and function of the associated tumor vasculature, has also been shown to be associated with chemotherapy therapy-resistance and biologically-aggressive malignant disease. Oxidative stress, commonly found in regions of intermittent hypoxia, has been implicated in regulation of glutathione metabolism, thus linking increased NPSH levels to tumor hypoxia. Therefore, it is also important to characterize both NPSH expression and its relationship to tumor hypoxia in tumors and other neoplastic tissues.

The heterogeneity of NPSH levels was examined in multiple biopsies obtained from patients with cervical carcinomas who were entered into a study investigating the activity of cellular oxidation and reduction levels (specifically, hypoxia) on the response to radical radiotherapy by Fyles, et al., (Oxygenation predicts radiation response and survival in patients with cervix cancer. Radiother. Oncol. 48:149-156 (1998)). The major findings from this study were that the intertumoral heterogeneity of the concentrations of GSH and cysteine exceeds the intratumoral heterogeneity, and that cysteine concentrations of approximately 21 mM were found in some samples, confirming an earlier report by Guichard, et al., (Glutathione and cysteine levels in human tumour biopsies. Br. J. Radiol. 134:63557-635561 (1990)). These levels of cysteine are much greater than those typically seen in tissue culture, suggesting that cysteine might exert a significant radioprotective activity in cervical carcinomas and possibly other types of cancer.

There is also extensive literature showing that elevated cellular glutathione levels can produce drug resistance in experimental models, due to drug detoxification or to the antioxidant activity of GSH. In addition, radiation-induced DNA radicals can be repaired non-enzymatically by GSH and cysteine, indicating a potential role for NPSH in radiation resistance. While cysteine is the more effective radioprotective agent, it is usually present in lower concentrations than GSH. Interestingly, under fully aerobic conditions, this radioprotective activity appears to be relatively minor, and NPSH compete more effectively with oxygen for DNA radicals under the hypoxic conditions that exist in some solid tumors, which might play a significant role in radiation resistance.

Radiotherapy has traditionally been a major treatment modality for cervical carcinomas. Randomized clinical trials (Rose, et al., Concurrent cisplatin-based radiotherapy and chemotherapy for locally advanced cervical cancer. New Engl. J. Med. 340:1144-1153 (1999)) show that patient outcome is significantly improved when radiation therapy is combined with cisplatin-based chemotherapy, and combined modality therapy is now widely being utilized in treatment regimens. It is important to establish the clinical relevance of GSH and cysteine levels to drug and radiation resistance because of the potential to modulate these levels using agents such as buthionine sulfoximine; an irreversible inhibitor of γ-glutanylcysteine synthetase that can produce profound depletion of GSH in both tumor and normal tissues. See, e.g., Bailey, et al., Phase I clinical trial of intravenous buthionine sulfoximine and melphalan: An attempt at modulation of glutathione. J. Clin. Oncol. 12:194-205 (1994). Evaluation of GSH concentrations have reported elevated tumor GSH relative to adjacent normal tissue, and intertumoral heterogeneity in GSH content. These findings are consistent with the idea that GSH could play a clinically significant role in drug resistance although it should be noted that relatively few studies have the sample size and follow up duration necessary to detect a significant relation between tumor GSH content and response to chemotherapy, hence there are no consistent clinical data to support this idea.

Koch and Evans (Cysteine concentrations in rodent tumors: unexpectedly high values may cause therapy resistance. Int. J. Cancer 67:661-667 (1996)) have shown that cysteine concentrations in established tumor cell lines can be much greater when these are grown as in vivo tumors, as compared to the in vitro values, suggesting that cysteine might play a more significant role in therapy resistance than previously considered. Although relatively few studies have reported on cysteine levels in human cancers, an earlier HPLC-based study of cervical carcinomas by Guichard, et al., (Glutathione and cysteine levels in human tumour biopsies. Br. J. Radiol. 134:63557-635561 (1990) reported cysteine concentrations greater than 1 mM in a significant number of cases. Thus, the fact that the variability in cysteine levels is greater than that for GSH suggests that these two thiols are regulated differently in tumors. By way of non-limiting example, the inhibition of γ-glutamylcysteine synthetase with the intravenous administration of buthionine sulfoximine (BSO) could result in elevated cellular levels of cysteine, due to the fact that the γ-glutamylcysteine synthetase is not being utilized for GSH de novo synthesis. Similar to GSH, cysteine possesses the ability to repair radiation-induced DNA radicals and cysteine also has the potential to detoxify cisplatin; a cytotoxic agent now routinely combined with radiotherapy to treat locally-advanced cervical carcinomas.

### Thioredoxin

Thioredoxin (THX) and glutaredoxin are members of the thioredoxin superfamily; they mediate disulfide exchange via their Cys-XI-X2-Cys active site. While glutaredoxins mostly reduce mixed disulfides containing glutathione, thioredoxins are involved in the maintenance of protein sulfhydryls in their reduced state via disulfide bond reduction. See, e.g., Print, W. A., et al., The role of the thioredoxin and glutaredoxin pathways in reducing protein disulfide bonds in the Escherichia coli cytoplasm. J. Biol. Chem. 272:15661-15667 (1996). The reduced form of thioredoxin is generated by the action of thioredoxin reductase; glutathione provides directly the reducing potential for regeneration of the reduced form of glutaredoxin.

There is a significant role of cellular thiols in regulation of a number of redox-sensitive transcription factors including, but not limited to, heat shock factor-1 (HSF-1), heat shock element-I (HSE-1), p53, activator protein 1(AP-1); the aforementioned proteins are activated under conditions of oxidative stress and subsequently translocated into the nucleus. See, e.g., Arrigo A. P., Gene expression and the thiol redox state. Free Rad. Biol. Med. 27:936-944 (1999). One of the key regulatory molecules in oxidative stress-induced cell activation is nuclear factor-κB (NF-κB) which is normally sequestered in the cytoplasm of non-stimulated cells and must be translocated into the nucleus to regulate activity or gene expression (e.g., those encoding cytokines and adhesion molecules). Also of particular interest is the role of redox factor 1 (Ref-1), a nuclear redox protein which is active in the regulation of DNA transcription. Ref-1 facilitates the binding of transcription factors to their respective DNA sequences by reduction of cysteine residues in their DNA binding domains. Thioredoxin plays a regulatory role in mediating this thiol-disulfide exchange by supplying reducing equivalents to Ref-1.

Various representative dithio-containing compounds of the present invention have been synthesized and purified. Additionally, disodium 2,2′-dithio-bis ethane sulfonate (also referred to in the literature as Tavocept™, dimesna, and BNP7787), a dithio-containing compound of the present invention, has been introduced into Phase I, Phase II, and Phase III clinical testing in patients, as well as in non-clinical testing, by the Assignee, BioNumerik Pharmaceuticals, Inc., with guidance provided by the Applicant of the instant invention. For example, data from recent Phase III clinical trials utilizing disodium 2,2′-dithio-bis ethane sulfonate (Tavocept™) will be undergoing review with the aim of further evaluating the ability of disodium 2,2′-dithio-bis ethane sulfonate to augment the anti-cancer activity of chemotherapeutic agents by increasing oxidative stress within tumor cells in a selective manner.

New formulations and methods of administration of agents such as disodium 2,2′-dithio-bis-ethane sulfonate in combination with one or more chemotherapeutic agents have now been discovered in connection with a human clinical study comprising a randomized, double-blind, placebo-controlled study with a 1:1 randomization. The Applicant of the present invention believes that further evaluation of the Phase III clinical study results will lend support for the ability of, e.g., disodium 2,2′-dithio-bis-ethane sulfonate to augment the anti-cancer activity of various chemotherapeutic agents in a selective manner while avoiding deleterious chemotherapeutic agent-induced effects on normal (i.e., non-cancerous) cells and tissues.

The present invention includes methods, formulations, and devices, including an effective amount of a dithio-containing compound of the present invention, which includes 2,2′-dithio-bis-ethane sulfonate, a pharmaceutically-acceptable salt thereof, an analog thereof, and the compounds of Formula (I). The methods, formulations, and devices may be administered: (i) in a stimulatory (i.e., inducing oxidative stress) or depletive (i.e., decreasing anti-oxidative capacity) manner to a cancer patient prior to the administration of an oxidative stress-inducing chemotherapeutic agent or agents in order to sensitize the neoplasm to enhance the tumor cytotoxicity of chemotherapy; (ii) in a therapeutic manner, as a cancer patient begins a chemotherapy cycle, in order to augment the activity of the oxidative stress induced by the chemotherapeutic agent or agents; and/or (iii) in a subsequent manner (i.e., after said chemotherapy cycle) in order to continue the induction or maintenance of the oxidative stress process in cancer cells and to prevent or mitigate any chemotherapy-associated side-effect(s). Additionally, the aforementioned compositions and formulations may be given in an identical manner to augment the anti-cancer activity of a cytotoxic agent by any clinically-beneficial mechanism(s).

The present invention additionally involves the use of the methods and the administration of the compositions and formulations described herein to a subject, optionally with or within a device, wherein the administration takes place as medically indicated in the subject prior to, concurrently or simultaneously, or following the administration of any chemotherapeutic agent or pharmaceutically active compound(s) by any route, dose, concentration, osmolarity, duration or schedule. Some of such routes, doses, concentrations, osmolarities, durations or schedules have been disclosed in U.S. patent application Ser. No. 11/638,193, entitled “Chemoprotective Methods and Compositions”, filed Dec. 13, 2006, the disclosure of which is hereby incorporated by reference in its entirety.

Various chemotherapeutic agents may be used in conjunction with, or as a part of, the methods described and claimed herein. Chemotherapeutic agents may include, for example, a fluropyrimidine; a pyrimidine nucleoside; a purine nucleoside; an antifolate, a platinum analog; an anthracycline/anthracenedione; an epipodophyllotoxin; a camptothecin; a hormone; a hormonal analog; an antihormonal; an enzyme, protein, peptide, or polyclonal or monoclonal antibody; a vinca alkaloid; a taxane; an epothilone; an antimicrotubule agent; an alkylating agent; an antimetabolite; a topoisomerase inhibitor; an aziridine-containing compound; an antiviral; or another cytotoxic and/or cytostatic agent. Fluropyrimidines include, for example, 5-fluorouracil (5-FU), S-1, capecitabine, ftorafur, 5′deoxyflurouridine, UFT, eniluracil, and the like. Pyrimidine nucleosides include, for example, cytarabine, deoxycytidine, 5-azacytosine, gemcitabine, 5-azadeoxycytidine, and the like. Purine nucleosides include, for example, fludarabine, 6-mercaptopurine, thioguanine, allopurinol, cladribine, and 2-chloro adenosine. Antifolates include, for example, methotrexate (MTX), pemetrexed (Alimta®), trimetrexate, aminopterin, methylene-10-deazaminopterin (MDAM), and the like. Platinum analogs include, for example, cisplatin, carboplatin, oxaliplatin, satraplatin, picoplatin, tetraplatin, platinum-DACH and analogs thereof. Anthracyclines/anthracenediones include, for example, doxorubicin, daunorubicin, epirubicin, and idarubicin. Epipodophyllotoxin derivatives include, for example, etoposide, etoposide phosphate and teniposide. Camptothecins include, for example, irinotecan, topotecan, 9-aminocamptothecin, 10,11-methylenedioxycamptothecin, karenitecin, 9-nitrocamptothecin, and TAS 103. Hormones and hormonal analogs may include, for example, (i) estrogens and estrogen analogs, including anastrazole, diethylstilbesterol, estradiol, premarin, raloxifene; progesterone, progesterone analogs and progestins, including progesterone, norethynodrel, esthisterone, dimesthisterone, megestrol acetate, medroxyprogesterone acetate, hydroxyprogesterone caproate, and norethisterone; (ii) androgens, including fluoxymesterone, methyltestosterone and testosterone; and (iii) adrenocorticosteroids, including dexamthasone, prednisone, cortisol, solumedrol, and the like. Antihormones include, for example, (i) antiestrogens, including: tamoxifen, fulvestrant, toremifene; aminoglutethimide, testolactone, droloxifene, and anastrozole; (ii) antiandrogens, including: bicalutamide, flutamide, nilutamide, and goserelin; (iii) antitestosterones, including: flutamide, leuprolide, and triptorelin; and (iv) adrenal steroid inhibitors including: aminoglutethimide and mitotane; and anti-leuteinizing hormones, including goserelin. Enzymes, proteins, peptides, polyclonal and/or monoclonal antibodies, may include, for example, asparaginase, cetuximab, erlotinib, bevacizumab, rituximab, gefitinib, trastuzumab, interleukins, interferons, leuprolide, pegasparanase, and the like. Vinca Alkaloids include, for example, vincristine, vinblastine, vinorelbine, vindesine, and the like. Taxanes include, for example, paclitaxel, docetaxel, and formulations and analogs thereof. Alkylating agents may include, for example, dacarbazine; procarbazine; temozolamide; thiotepa; nitrogen mustards (e.g., mechlorethamine, chlorambucil, L-phenylalanine mustard, melphalan, and the like); oxazaphosphorines (e.g., ifosphamide, cyclophosphamide, mefosphamide, perfosfamide, trophosphamide and the like); alkyl sulfonates (e.g., busulfan); and nitrosoureas (e.g., carmustine, lomustine, semustine and the like). Epothilones include, for example, epothilones A-E. Antimetabolites include, for example, tomudex and methotrexate, trimetrexate, aminopterin, pemetrexid, MDAM, 6-mercaptopurine, and 6-thioguanine. Topoisomerase inhibitors include, for example, irinotecan, topotecan, karenitecin, amsacrine, etoposide, etoposide phosphate, teniposide, and doxorubicin, daunorubicin, and other analogs. Antiviral agents include, for example, acyclovir, valacyclovir, ganciclovir, amantadine, rimantadine, lamivudine, and zidovudine. Monoclonal antibody agents include, for example, bevacizumab, trastuzumab, rituximab, and the like, as well as growth inhibitors such as erlotinib, and the like. In general, cytostatic agents are mechanism-based agents that slow the progression of neoplastic disease.

In one embodiment of the present invention, the administration of an effective amount of a formulation comprising a dithio-containing compound of the present invention, which includes 2,2′-dithio-bis-ethane sulfonate, a pharmaceutically-acceptable salt thereof, an analog thereof, and the compounds of Formula (I), elicits an augmentation of the anti-cancer activity of chemotherapeutic agents by a prevention and/or reduction in the normal increase or responsiveness in the concentration and metabolism of Glutathione/cysteine and other physiological cellular thiols; these antioxidants and enzymes are increased in concentration and activity, respectively, in response to intracellular oxidative stress which may be induced by exposure to chemotherapeutic agents in tumor cells, thus increasing chemotherapeutic agent efficacy and decreasing tumor-mediated drug resistance.

In another embodiment, the administration of an effective amount of a formulation comprising a dithio-containing compound of the present invention, which includes 2,2′-dithio-bis-ethane sulfonate, a pharmaceutically-acceptable salt thereof, an analog thereof, and the compounds of Formula (I), elicits an augmentation of the anti-cancer activity of chemotherapeutic agents by thioredoxin inactivation by said dithio-containing compounds, thereby increasing apoptotic sensitivity and decreasing mitogenic/cellular replication signaling.

In yet another embodiment of the present invention, the administration of an effective amount of a formulation comprising a dithio-containing compound of the present invention, which includes 2,2′-dithio-bis-ethane sulfonate, a pharmaceutically-acceptable salt thereof, an analog thereof, and the compounds of Formula (I), elicits an augmentation of the anti-cancer activity of chemotherapeutic agents by a key metabolite of, e.g., disodium 2,2′-dithio-bis-ethane sulfonate (dimesna), said metabolite known as 2-mercapto ethane sulfonate sodium (mesna) which possesses intrinsic cytotoxic activity (i.e., causes apoptosis) in some tumors by an, as yet, unknown mechanism.

In one embodiment, the administration of an effective amount of a formulation comprising a dithio-containing compound of the present invention, which includes 2,2′-dithio-bis-ethane sulfonate, a pharmaceutically-acceptable salt thereof, an analog thereof, and the compounds of Formula (I), elicits an augmentation of the anti-cancer activity of chemotherapeutic agents by reducing oxidative potential or by compromising the anti-oxidative response of tumor cells, thus enhancing the oxidative biological state and oxidative damage in tumor cells exposed to chemotherapy and increasing the associated cytotoxicity/apoptosis of the chemotherapy agents. By reducing or compromising the total anti-oxidative capacity or responsiveness of tumor cells, a marked increase in cellular apoptosis can be achieved. It is believed by the Applicant of the present invention that this is a key anti-cancer augmentation mechanism of action that may act synergistically with other mechanisms of anti-cancer augmentation of the dithio-containing compounds of the present invention.

In another embodiment, an effective amount of a formulation comprising a dithio-containing compound of the present invention, which includes 2,2′-dithio-bis-ethane sulfonate, a pharmaceutically-acceptable salt thereof, an analog thereof, and the compounds of Formula (I), may be administered in a preventative (i.e., prophylactic) manner to a cancer patient prior to chemotherapy in order to “sensitize” the cancer.

In yet another embodiment of the present invention, an effective amount of a formulation comprising a dithio-containing compound of the present invention, which includes 2,2′-dithio-bis-ethane sulfonate, a pharmaceutically-acceptable salt thereof, an analog thereof, and the compounds of Formula (I), may be administered therapeutically once a cancer patient begins a chemotherapy cycle to help augment the activity of the chemotherapeutic agent(s).

In one embodiment, the administration of an effective amount of a formulation comprising a dithio-containing compound of the present invention, which includes 2,2′-dithio-bis-ethane sulfonate, a pharmaceutically-acceptable salt thereof, an analog thereof, and the compounds of Formula (I), may be administered after said chemotherapy cycle to continue the sensitization process and to prevent or mitigate the chemotherapy-agent(s) associated side-effect(s).

In another embodiment, the administration of an effective amount of a formulation comprising a dithio-containing compound of the present invention, which includes 2,2′-dithio-bis-ethane sulfonate, a pharmaceutically-acceptable salt thereof, an analog thereof, and the compounds of Formula (I), may be administered concurrently or following the chemotherapy cycle to augment anti-cancer activity of a cytotoxic agent.

In yet another embodiment of the present invention, an effective amount of a dithio-containing compound of the present invention, which includes 2,2′-dithio-bis-ethane sulfonate, a pharmaceutically-acceptable salt thereof, an analog thereof, and the compounds of Formula (I), may include, for example, a range from about 0.01 g/m² to about 100 g/m². Additional effective doses may include, for example, from about 0.1 g/m² to about 90 g/m²; about 1.0 g/m² to about 80 g/m²; about 4.0 g/m² to about 70 g/m² about 5.0 g/m² to about 60 g/m²; about 10 g/m² to about 50 g/m²; about 15 g/m² to about 25 g/m²; about 4 g/m²; about 8 g/m²; about 12 g/m²; about 18 g/m²; about 28 g/m²; about 35 g/m²; and about 41 g/m². Other amounts within these ranges may also be used. The aforementioned dithio-containing compounds of the present invention will be administered to a subject who has received, is currently receiving, or will receive one or more chemotherapeutic agents.

In one preferred embodiment, a dithio-containing compound of the present invention, which includes 2,2′-dithio-bis-ethane sulfonate, a pharmaceutically-acceptable salt thereof, an analog thereof, and the compounds of Formula (I), is administered at a concentration of about 100 mg/mL. In another preferred embodiment a dithio-containing compound of the present invention is infused over about 45 minutes. In yet another preferred embodiment a dithio-containing compound of the present invention is administered at a concentration of about 100 mg/mL over a period of about 45 minutes. The aforementioned dithio-containing compounds of the present invention will be administered to a subject who has received, is currently receiving, or will receive one or more chemotherapeutic agents.

In another embodiment, a dithio-containing compound of the present invention, which includes 2,2′-dithio-bis-ethane sulfonate, a pharmaceutically-acceptable salt thereof, an analog thereof, and the compounds of Formula (I), may be administered, for example, at an infusion rate of about 0.1 g/min. to about 4.6 g/min. Additional infusion rates include, for example, about 0.2 g/min to about 2.0 g/min.; about 0.2 g/min. to about 4.0 g/min.; about 0.25 g/min. to about 3.0 g/min., about 0.3 g/min. to about 2.5 g/min.; about 0.35 g/min. to about 2.0 g/min.; about 0.4 g/min. to about 1.5 g/min.; about 0.45 g/min. to about 1.4 g/min.; about 0.5 g/min. to about 1.3 g/min.; about 0.55 g/min. to about 1.3 g/min.; about 0.6 g/min. to about 1.2 g/min.; about 0.55 g/min. to about 1.2 g/min.; about 0.6 g/min. to about 1.1 g/min.; about 0.65 g/min. to about 1.0 g/min. Other amounts within these ranges may also be used. The infusion rate can be calculated by those skilled in the art based on the desired dose per mass, Body Surface Area (BSA) of the subject and infusion time. For example, a dose of about 18.4 g/m², in a patient with a BSA of 1.7 m², infused over 45 minutes would have an infusion rate of about 0.7 g/minute. The aforementioned dithio-containing compounds of the present invention will be administered to a subject who has received, is currently receiving, or will receive one or more chemotherapeutic agents.

In yet another embodiment, a dithio-containing compound of the present invention, which includes 2,2′-dithio-bis-ethane sulfonate, a pharmaceutically-acceptable salt thereof, an analog thereof, and the compounds of Formula (I), is administered, for example, at about 1.0 mg/mL/min. to about 50 mg/mL/min. Additional dosing may include, for example, from about 2.0 mg/mL/min. to about 20 mg/mL/min.; about 1.5 mg/mL/min. to about 40 mg/mL/min.; about 2.0 mg/mL/min. to about 35 mg/mL/min.; about 2.5 mg/mL/min. to about 30 mg/mL/min.; about 3.0 mg/mL/min. to about 25 mg/mL/min.; about 3.5 mg/mL/min. to about 20 mg/mL/min.; about 4.0 mg/mL/min. to about 15 mg/mL/min.; about 4.5 mg/mL/min.; about 5.0 mg/mL/min.; about 5.5 mg/mL/min.; about 6.0 mg/mL/min.; about 6.5 mg/mL/min.; about 7.0 mg/mL/min.; about 7.5 mg/mL/min.; about 8.0 mg/mL/min.; about 8.5 mg/mL/min.; about 9.0 mg/mL/min.; about 9.5 mg/mL/min.; about 10 mg/mL/min.; about 11 mg/mL/min.; about 12 mg/mL/min.; about 13 mg/mL/min.; and about 14 mg/mL/min. Other amounts approximating these ranges may also be utilized. The mg/mL/min dosing schedule can be calculated by those skilled in the art based on a desired dose per mass, BSA of the subject, infusion time, and desired concentration. For example, a dose of about 18.4 g/m², in a patient with a BSA of about 1.7 m², infused over 45 minutes at a concentration of 100 mg/mL would be about 7 mg/mL/min. The aforementioned anti-cancer augmentation dithio-containing compounds of the present invention will be administered to a subject who has received, is currently receiving, or will receive one or more chemotherapeutic agents.

In one preferred embodiment, the method of administration comprises administration of a dithio-containing compound of the present invention, which includes 2,2′-dithio-bis-ethane sulfonate, a pharmaceutically-acceptable salt thereof, an analog thereof, and the compounds of Formula (I), in a composition that is hyperosmotic relative to the patient's plasma or serum osmolarity. In one embodiment, for example, the compound is administered in a composition having an osmolarity of about 0.1- to about 5-times the osmolarity of the normal plasma or serum osmolarity in a subject. In another embodiment, the compound is administered in a composition having an osmolarity of about 2- to about 4-times the osmolarity of the normal plasma or serum osmolarity in a subject. In yet other embodiments, the compound is administered in a composition having an osmolarity of about 1-; about 2-; about 3-; about 4-; or about 5-times the osmolarity of the normal plasma or serum osmolarity in a subject. The normal range of human plasma osmolarity ranges from approximately 280 mOsm to approximately 320 mOsm. The aforementioned dithio-containing compounds of the present invention will be administered to a subject who has received, is currently receiving, or will receive one or more chemotherapeutic agents.

In one embodiment of the present invention, a dithio-containing compound of the present invention is a pharmaceutically-acceptable disodium salt. In various other embodiments, a dithio-containing compound of the present invention is/are a pharmaceutically-acceptable salt(s) which include, for example: (i) a monosodium salt; (ii) a sodium potassium salt; (iii) a dipotassium salt; (iv) a calcium salt; (v) a magnesium salt; (vi) a manganese salt; (vii) a monopotassium salt; and (viii) an ammonium salt. It should be noted that mono- and di-potassium salts of 2,2′-dithio-bis-ethane sulfonate and/or an analog thereof are administered to a subject if the total dose of potassium administered at any given point in time is not greater than 100 Meq. and the subject is not hyperkalemic and does not have a condition that would predispose the subject to hyperkalemia (e.g., renal failure).

By way of non-limiting example, disodium 2,2′-dithio-bis-ethane sulfonate (also referred to in the literature as dimesna, Tavocept™, and BNP7787) is a known compound and can be manufactured by methods known in the art. See, e.g., J. Org. Chem. 26:1330-1331 (1961); J. Org. Chem. 59:8239 (1994). In addition, various salts of 2,2′-dithio-bis-ethane sulfonate, as well as other dithioethers may also be synthesized as outlined in U.S. Pat. No. 5,808,160, U.S. Pat. No. 6,160,167 and U.S. Pat. No. 6,504,049. Compounds of Formula (I) may be manufactured as described in Published U.S. Patent Application 2005/0256055. The disclosures of these patents, patent applications, and published patent applications are incorporated herein by reference, in their entirety.

In another embodiment, the method of administration further comprises the step of administering one or more chemotherapeutic agents. The administration of a dithio-containing compound of the present invention may be prior to, immediately prior to, during, immediately subsequent to, or subsequent to the administration of one or more chemotherapeutic agents.

Chemotherapeutic agents may be prepared and administered to subjects using methods known within the art. For example, paclitaxel may be prepared using methods described in U.S. Pat. Nos. 5,641,803, 6,506,405, and 6,753,006 and is administered as known in the art (see, e.g., U.S. Pat. Nos. 5,641,803, 6,506,405, and 6,753,006). Paclitaxel may be prepared for administration in a dose in the range of about 50 mg/m² and about 275 mg/m². Preferred doses include about 80 mg/m², about 135 mg/m² and about 175 mg/m².

Docetaxel may be prepared using methods described in U.S. Pat. No. 4,814,470 and is administered as known in the art (see, e.g., U.S. Pat. Nos., 4,814,470, 5,438,072, 5,698,582, and 5,714,512). Docetaxel may be prepared for administration in a dose in the range of about 30 mg/m² and about 100 mg/m². Preferred doses include about 55 mg/m², about 60 mg/m², about 75 mg/m², and about 100 mg/m².

Cisplatin may be prepared using methods described in U.S. Pat. Nos. 4,302,446, 4,322,391, 4,310,515, and 4,915,956 and is administered as known in the art (see, e.g., U.S. Pat. Nos. 4,177,263, 4,310,515, 4,451,447). Cisplatin may be prepared for administration in a dose in the range of about 30 mg/m² and about 120 mg/m² in a single dose or 15 mg/m² and about 20 mg/m² daily for five days. Preferred doses include about 50 mg/m², about 75 mg/m² and about 100 mg/m².

Carboplatin may be prepared using methods described in U.S. Pat. No. 4,657,927 and is administered as known in the art (see, e.g., U.S. Pat. No. 4,657,927). Carboplatin may be prepared for administration in a dose in the range of about 20 mg/kg and about 200 mg/kg. Preferred doses include about 300 mg/m² and about 360 mg/m². Other dosing may be calculated using a formula according to the manufacturer's instructions.

Oxaliplatin may be prepared using methods described in U.S. Pat. Nos. 5,290,961, 5,420,319, 5,338,874 and is administered as known in the art (see, e.g., U.S. Pat. No. 5,716,988). Oxaliplatin may be prepared for administration in a dose in the range of about 50 mg/m² and about 200 mg/m². Preferred doses include about 85 mg/m² and about 130 mg/m².

In another embodiment, the method comprises one or more additional hydration step(s). Such hydration may serve, e.g., to replace or increase internal fluid levels. For example, saline hydration may include administration of about 250 mL to about 1000 mL of 0.9% saline solution administered over about 1 hour to about 2 hours. Other forms of hydration, including hypertonic (e.g., 3% sodium chloride) or hypotonic (e.g., 0.45 M sodium chloride or Dextrose 5% in Water or Ringer's lactate) solutions that are commercially available for parenteral administration, may be used in lieu of, or in combination with, or in addition to saline hydration as dictated by the patient's medical condition.

In yet another embodiment of the present invention, the method comprises an additional step of administering one or more pre-therapy medication(s). Pre-medications include, for example, antihistamines, steroids, antimetics, and 5-HT3 antagonists. Antihistamines may include, for example, diphenhydramine, clemastine, cimetidine, ranitidine and famotidine. Steroids may include, for example, corticosteroids, including hydrocortisone, dexamethasone, prednisolone and prednisone. Antiemetics may include, for example, prochloroperazine, metoclopramide, and dimenhydrinate. 5-HT3 antagonists may include, for example, ondansetron, dolasetron, and granisetron. Other pre-therapy drugs may include, for example, diazepam congeners, gabapentin and amitryptiline. Pre-therapy may be administered at least one day prior to chemotherapy, prior to each chemotherapy treatment, immediately prior to each chemotherapy treatment, concomitantly with or simultaneously during chemotherapy treatment, immediately subsequent to chemotherapy, subsequent to chemotherapy, and/or according to methods known within the art and in accordance with the patient's medical condition.

In one embodiment, a dithio-containing compound of the present invention, which includes 2,2′-dithio-bis-ethane sulfonate, a pharmaceutically-acceptable salt thereof, an analog thereof, and the compounds of Formula (I), and one or more chemotherapeutic agents, are administered to a subject in need of treatment for one or more cancers. Said subject may be a human. Said cancer or cancers may be human cancers, which may include, for example, one or more cancers of the: ovary, breast, lung, esophagus, bladder, stomach, pancreas, liver (e.g., bile ducts, gall bladder, and Ampulla of Vater), testes, germ cell, bone, cartilage, head, neck, oral mucosa, colorectal area, anus, kidney, uroepithelium, central nervous system, prostate, endometrium, cervix, uterus, fallopian tube, peripheral nervous system, and various other cancers including melanoma, mesothelioma, myeloma, lymphoma, leukemia, and Kaposi's sarcoma.

The dosage forms, formulations, devices and/or compositions of the present invention may be formulated for periodic administration, including: at least one administration in an approximately 24 hour period; at least one administration in an approximately 48 hour period; at least about once every three days; at least about once every four days; at least about once every five days; at least about once every six days; at least about once a week; at least about once every 1.5 weeks or less; at least about once every 2 weeks or less; at least about once every 2.5 weeks or less; at least about once every 3 weeks or less; at least about once every 3.5 weeks or less; at least about once every 4 weeks or less; at least about once every 5 weeks or less; at least once at any time interval between one day and five weeks; or at least once at a time interval of more than every 5 weeks.

In one preferred embodiment, the composition of the invention comprises a dithio-containing compound of the present invention, which includes 2,2′-dithio-bis-ethane sulfonate, a pharmaceutically-acceptable salt thereof, an analog thereof, and the compounds of Formula (I), at about 100 mg/mL to about 200 mg/mL or, alternately, about 600 mOsm/L to about 1,800 mOsm/L. The composition may also include one or more chemotherapeutic agents.

In certain of the methods of the invention, as well as in the uses of the compositions and formulations of the invention, the chemoprotective agent may be administered in conjunction with one or more chemotherapeutic agent, wherein each course being of a specified period dependent upon the specific chemotherapeutic agent or agents utilized. In conjunction with the inventions described and claimed herein, the treatment regimens may be comprised, for example, of two or more treatment courses, of five or more treatment courses, of six or more treatment courses, of seven or more treatment courses, of eight or more treatment courses, or of nine or more treatment courses. The treatment courses may also be continuous. By way of non-limiting example, the chemotherapeutic agent may be a taxane chemotherapeutic agent, such as paclitaxel or docetaxel, which may also be administered in a course of therapy in combination with another chemotherapeutic agent, for example, a platinum chemotherapeutic agent (e.g., cisplatin or carboplatin).

The compositions and formulations of the present invention, alone or in combination with one or more chemotherapeutic agents, and instructions for their use, may be included in a form of packs or kits. Thus, the invention also includes kits comprising the compositions, formulations, and/or devices described herein with instructions for use. For example, a kit may comprise a dithio-containing compound of the present invention and instructions for administration. Kits may additionally comprise one or more chemotherapeutic agents with instructions for their use. Kits may also additionally comprise one or more pre-treatments as described herein and instructions for their use.

In general, the compositions and formulations of the present invention are administered once a day, wherein a chemotherapeutic agent is administered at 1 day to 5 week intervals, or any times in between, or longer than 5 week intervals as described herein. By way of non-limiting example, a dithio-containing compound of the present invention which possesses anti-cancer augmentation activity may be administered prior to, concomitantly with, or subsequent to the administration of the chemotherapeutic agent or agents.

In one example, a course of therapy may include a single dose of paclitaxel (e.g., 175 mg/m²) administered intravenously over 3 hours, pre-cisplatin saline hydration for 1 hour, immediately followed by a single dose of a dithio-containing compound of the present invention (in a formulation having the concentration and/or osmolarity set forth herein, and/or administered at a rate set forth herein) administered intravenously over about 45 minutes, a single dose of cisplatin (e.g., 75 mg/m²) administered intravenously over 1 hour and subsequently post-cisplatin saline hydration for 1.5 hours. As previously discussed, the methods of the present invention may be carried out, and the formulations of the invention used, with only one chemotherapeutic agent (e.g., a taxane or a platinum chemotherapeutic agent) or with more than one chemotherapeutic agent.

As noted herein, the methods of the invention may also be carried out, and the formulations of the invention also used, in conjunction with one or more pre-medications. Pre-medications may be administered at least one day prior to chemotherapy, prior to each chemotherapy treatment, immediately prior to each chemotherapy treatment, concomitantly with or simultaneously during chemotherapy treatment, immediately subsequent to chemotherapy, subsequent to chemotherapy, and/or according to methods known within the art and in accordance with the patient's medical condition. Pre-medications may be administered according to the manufacture's instructions. Saline hydration may include, for example, administration of about 250 mL to about 1000 mL of 0.9% saline solution administered over about 1 hour to about 2 hours. Other forms of hydration, including hypertonic (e.g., 3% sodium chloride) or hypotonic (e.g., 0.45% sodium chloride or Dextrose 5% in Water or Ringer's lactate) solutions that are commercially available for parenteral administration, may be used in lieu of, or in combination with, or in addition to saline hydration as dictated by the patient's medical condition. Hydration steps can be added prior to the administration of paclitaxel, after administration of a dithio-containing compound, prior to the administration of cisplatin, and/or after the administration of cisplatin.

Aspects of the present invention also include controlled delivery or other doses, dosage forms, formulations, compositions and/or devices containing a dithio-containing compound of the present invention, which includes 2,2′-dithio-bis-ethane sulfonate, a pharmaceutically-acceptable salt thereof, an analog thereof, and the compounds of Formula (I), as well as one or more chemotherapeutic agents, for example, various doses and dosage forms for: (i) oral (e.g., tablet, suspension, solution, gelatin capsule (hard or soft), sublingual, dissolvable tablet, troche, and the like), or with sublingual administration which avoids first-pass metabolism through the liver (i.e., the cytochrome P₄₅₀ oxidase system); (ii) injection (e.g., subcutaneous administration, intradermal administration, subdermal administration, intramuscular administration, depot administration, intravenous administration, intra-arterial administration, and the like), wherein the administration may occur by, e.g., injection delivery, delivery via parenteral bolus, slow intravenous injection, and intravenous drip, and infusion devices (e.g., implantable infusion devices, both active and passive); (iii) intra-cavitary (e.g., into the intrapleural, intraperitoneal, intravesicular, and/or intrathecal spaces); (iv) per rectum (e.g., suppository, retention enema); and (v) topical administration routes to subjects as treatment for various cancers.

Examples of dosage forms suitable for injection of the compounds and formulations of the present invention include delivery via bolus such as single or multiple or continuous or constant administrations by intravenous injection, subcutaneous, subdermal, and intramuscular administration. These forms may be injected using syringes, pens, jet injectors, and internal or external pumps, with vascular or peritoneal access, for example. Syringes come in a variety sizes including 0.3, 0.5, 1, 2, 5, 10, 25 and 50 mL capacity. Needleless jet injectors are also known in the art and use a pressurized air to inject a fine spray of solution into the skin. Pumps are also known in the art. The pumps are connected by flexible tubing to a catheter, which is inserted into the tissue just below the skin. The catheter is left in place for several days at a time. The pump is programmed to dispense the necessary amount of solution at the proper times.

Examples of infusion devices for compounds and formulations of the present invention include infusion pumps containing a dithio-containing compound of the present invention to be administered at a desired rate and amount for a desired number of doses or steady state administration, and include implantable drug pumps.

Examples of implantable infusion devices for compounds and formulations of the invention include any solid form or liquid form in which the active agent is a solution, suspension or encapsulated within or dispersed throughout a biodegradable polymer or synthetic polymer, for example, silicone, polypropylene, silicone rubber, silastic or similar polymer.

Examples of controlled release drug formulations useful for delivery of the compounds and formulations of the invention are found in, for example, Sweetman, S. C. (Ed.)., The Complete Drug Reference, 33rd Edition, Pharmaceutical Press, Chicago, 2483 pp. (2002); Aulton, M. E. (Ed.), Pharmaceutics: The Science of Dosage Form Design. Churchill Livingstone, Edinburgh, 734 pp. (2000); and, Ansel, H. C., Allen, L. V. and Popovich, N. G., Pharmaceutical Dosage Forms and Drug Delivery Systems, 7th Ed., Lippincott, 676 pp. (1999). Excipients employed in the manufacture of drug delivery systems are described in various publications known to those skilled in the art including, for example, Kibbe, E. H., Handbook of Pharmaceutical Excipients, 3rd Ed., American Pharmaceutical Association, Washington, 665 pp. (2000).

Further examples of dosage forms of the present invention primarily utilized with oral administration, include but are not limited to, modified-release (MR) dosage forms including delayed-release (DR) forms; prolonged-action (PA) forms; controlled-release (CR) forms; extended-release (ER) forms; timed-release (TR) forms; and long-acting (LA) forms. As previously stated, these formulations are often used with orally administered dosage forms, however these terms may be applicable to any of the dosage forms, formulations, compositions and/or devices described herein. These formulations delay and control total drug release for some time after drug administration, and/or drug release in small aliquots intermittently after administration, and/or drug release slowly at a controlled rate governed by the delivery system, and/or drug release at a constant rate that does not vary, and/or drug release for a significantly longer period than usual formulations.

Modified-release dosage forms of the present invention include dosage forms having drug release features based on time, course, and/or location which are designed to accomplish therapeutic or convenience objectives not offered by conventional or immediate-release forms. See, e.g., Bogner, R. H., Bioavailability and bioequivalence of extended-release oral dosage forms. U.S. Pharmacist 22 (Suppl.):3-12 (1997). Extendedrelease dosage forms of the invention include, for example, as defined by the FDA, a dosage form that allows a reduction in dosing frequency to that represented by a conventional dosage form, e.g., a solution or an immediate-release dosage form.

For example, one embodiment provides extended-release formulations containing a dithio-containing compound of the present invention for parenteral administration. Extended rates of activity of a dithio-containing compound of the present invention following injection may be achieved in a number of ways, including the following: crystal or amorphous dithio-containing compound forms having prolonged dissolution characteristics; slowly dissolving chemical complexes of dithio-containing compound formulations; solutions or suspensions of a dithio-containing compound of the present invention in slowly absorbed carriers or vehicles (e.g., oleaginous); increased particle size of a dithio-containing compound of the present invention, in suspension; or, by injection of slowly eroding microspheres of said dithio-containing compounds (see, e.g., Friess, W., et al., Insoluble collagen matrices for prolonged delivery of proteins. Pharmaceut. Dev. Technol. 1:185-193 (1996)). For example, the duration of action of the various forms of insulin is based in part on its physical form (i.e., amorphous or crystalline), complex formation with added agents, and its dosage form (i.e., solution or suspension).

An acetate, phosphate, citrate, bicarbonate, glutamine or glutamate buffer may be added to modify pH of the final composition. Optionally a carbohydrate or polyhydric alcohol tonicifier and, a preservative selected from the group consisting of m-cresol, benzyl alcohol, methyl, ethyl, propyl and butyl parabens and phenol may also be added. Water for injection, tonicifying agents such as sodium chloride, as well as other excipients, may also be present, if desired. For parenteral administration, formulations may be isotonic or substantially isotonic to avoid irritation and pain at the site of administration. Alternatively, formulations for parenteral administration may also be hyperosmotic relative to normal mammalian plasma, as described herein.

The terms buffer, buffer solution and buffered solution, when used with reference to hydrogen-ion concentration or pH, refer to the ability of a solute/solvent system, particularly an aqueous solution, to resist a change in pH with the addition of acid or alkali, or upon dilution with a solvent, or both. Characteristic of buffered solutions, which undergo small changes of pH on addition of acid or base, is the presence either of a weak acid and a salt of the weak acid, or a weak base and a salt of the weak base. An example of the former system is acetic acid and sodium acetate. The change of pH is slight as long as the amount of hydroxyl ion added does not exceed the capacity of the buffer system to neutralize it. The buffer used in the practice of the present invention is selected from any of the following, for example, an acetate, phosphate, citrate, bicarbonate, glutamine, or glutamate buffer, with the most preferred buffer being a phosphate buffer.

Carriers or excipients can also be used to facilitate administration of the compositions and formulations of the invention. Examples of carriers and excipients include calcium carbonate, calcium phosphate, various sugars such as lactose, glucose, or sucrose, or types of starch, cellulose derivatives, gelatin, polyethylene glycols, and physiologically compatible solvents.

A stabilizer may be included in the formulations of the invention, but will generally not be needed. If included, however, a stabilizer useful in the practice of the invention is a carbohydrate or a polyhydric alcohol. The polyhydric alcohols include such compounds as sorbitol, mannitol, glycerol, xylitol, and polypropylene/ethylene glycol copolymer, as well as various polyethylene glycols (PEG) of molecular weight 200, 400, 1450, 3350, 4000, 6000, and 8000). The carbohydrates include, for example, mannose, ribose, trehalose, maltose, inositol, lactose, galactose, arabinose, or lactose.

The United States Pharmacopeia (USP) states that anti-microbial agents in bacteriostatic or fungistatic concentrations must be added to preparations contained in multiple dose containers. They must be present in adequate concentration at the time of use to prevent the multiplication of microorganisms inadvertently introduced into the preparation while withdrawing a portion of the contents with a hypodermic needle and syringe, or using other invasive means for delivery, such as pen injectors. Antimicrobial agents should be evaluated to ensure compatibility with all other components of the formulation, and their activity should be evaluated in the total formulation to ensure that a particular agent that is effective in one formulation is not ineffective in another. It is not uncommon to find that a particular agent will be effective in one formulation but not effective in another formulation.

A preservative is, in the common pharmaceutical sense, a substance that prevents or inhibits microbial growth and may be added to a pharmaceutical formulation for this purpose to avoid consequent spoilage of the formulation by microorganisms. While the amount of the preservative is not great, it may nevertheless affect the overall stability of the dithio-containing compound of the present invention. Preservatives include, for example, benzyl alcohol and ethyl alcohol. While the preservative for use in the practice of the invention can range from 0.005 to 1.0% (w/v), the preferred range for each preservative, alone or in combination with others, is: benzyl alcohol (0.1-1.0%), or m-cresol (0.1-0.6%), or phenol (0.1-0.8%) or combination of methyl (0.05-0.25%) and ethyl or propyl or butyl (0.005%-0.03%) parabens. The parabens are lower alkyl esters of para-hydroxybenzoic acid. A detailed description of each preservative is set forth in “Remington's Pharmaceutical Sciences” as well as Pharmaceutical Dosage Forms: Parenteral Medications, Vol. 1, Avis, et al. (1992). For these purposes, the 2,2′-dithio-bis-ethane sulfonate, a pharmaceutically-acceptable salt thereof, an analog thereof, and/or a compound of Formula (I), may be administered parenterally (including subcutaneous injections, intravenous, intramuscular, intradermal injection or infusion techniques) in dosage unit formulations containing conventional non-toxic pharmaceutically acceptable carriers, adjuvants, and vehicles. In addition, formulations of the present invention designed for parenteral administration must be stable, sterile, pyrogen-free, and possess particulate levels and size within accepted levels.

If desired, the parenteral formulation may be thickened with a thickening agent such as a methylcellulose. The formulation may be prepared in an emulsified form, either water in oil or oil in water. Any of a wide variety of pharmaceutically-acceptable emulsifying agents may be employed including, for example, acacia powder, a non-ionic surfactant, or an ionic surfactant.

It may also be desirable to add suitable dispersing or suspending agents to the pharmaceutical formulation. These may include, for example, aqueous suspensions such as synthetic and natural gums, e.g., tragacanth, acacia, alginate, dextran, sodium carboxymethylcellulose, methylcellulose, polyvinyl-pyrrolidone, or gelatin.

It is possible that other ingredients may be present in the parenteral pharmaceutical formulation of the invention. Such additional ingredients may include wetting agents, oils (e.g., a vegetable oil such as sesame, peanut, or olive), analgesic agents, emulsifiers, antioxidants, bulking agents, tonicity modifiers, metal ions, oleaginous vehicles, proteins (e.g., human serum albumin, gelatin, or proteins) and a zwitterion (e.g., an amino acid such as betaine, taurine, arginine, glycine, lysine, or histidine). Such additional ingredients, of course, should not adversely affect the overall stability of the pharmaceutical formulation of the present invention.

Containers and kits are also a part of a composition and may be considered a component. Therefore, the selection of a container is based on a consideration of the composition of the container, as well as of the ingredients, and the treatment to which it will be subjected.

Suitable routes of parenteral administration include intramuscular, intravenous, subcutaneous, intraperitoneal, subdermal, intradermal, intraarticular, intrathecal, and the like. Mucosal delivery is also permissible. The dose and dosage regimen will depend upon the weight, health, disease type, and degree of disease severity within the subject. Regarding pharmaceutical formulations, see, Pharmaceutical Dosage Forms: Parenteral Medications, Vol. 1, 2nd ed., Avis et al., Eds., Marcel Dekker, New York, N.Y. (1992).

In addition to the above means of achieving extended drug action, the rate and duration of delivery of a dithio-containing compound of the present invention, as well as one or more chemotherapeutic agents may be controlled by, e.g., using mechanically controlled drug infusion pumps.

The present invention, in part, provides infusion dose delivery formulations and devices, including but not limited to, implantable infusion devices for delivery of compositions and formulations of the invention. Implantable infusion devices may employ inert material such as the biodegradable polymers described above or synthetic silicones, for example, cylastic, silicone rubber or other commercially-available polymers manufactured and approved for such uses. The polymer may be loaded with a dithio-containing compound of the present invention and any excipients. Implantable infusion devices may also comprise the coating of, or a portion of, a medical device wherein the coating comprises the polymer loaded with a dithio-containing compound of the present invention, one or more chemotherapeutic agents, and any excipient. Such an implantable infusion device may be prepared as disclosed in U.S. Pat. No. 6,309,380 by coating the device with an in vivo biocompatible and biodegradable or bioabsorbable or bioerodable liquid or gel solution containing a polymer with the solution comprising a desired dosage amount of a dithio-containing compound of the present invention, one or more chemotherapeutic agents, and any excipients. The solution is converted to a film adhering to the medical device thereby forming the implantable dithio-containing compound-deliverable medical device.

An implantable infusion device may also be prepared by the in situ formation of a dithio-containing compound of the present invention, containing a solid matrix (as disclosed in U.S. Pat. No. 6,120,789, the disclosure of which is hereby incorporated by reference, in its entirety) and one or more chemotherapeutic agents. Implantable infusion devices may be passive or active. An active implantable infusion device may comprise a dithio-containing compound reservoir, a means of allowing the dithio-containing compound to exit the reservoir, for example a permeable membrane, and a driving force to propel the dithio-containing compound from the reservoir. The reservoir of the aforementioned active implantable infusion device may also contain one or more chemotherapeutic agents. Such an active implantable infusion device may additionally be activated by an extrinsic signal, such as that disclosed in WO 02/45779, wherein the implantable infusion device comprises a system configured to deliver a dithio-containing compound of the present invention and one or more chemotherapeutic agents, comprising an external activation unit operable by a user to request activation of the implantable infusion device, including a controller to reject such a request prior to the expiration of a lockout interval. Examples of an active implantable infusion device include implantable drug pumps. Implantable drug pumps include, for example, miniature, computerized, programmable, refillable drug delivery systems with an attached catheter that inserts into a target organ system, usually the spinal cord or a vessel. See, Medtronic Inc. Publications: UC9603124EN NP-2687, 1997; UC199503941b EN NP-2347 182577-101, 2000; UC199801017a EN NP3273a 182600-101, 2000; UC200002512 EN NP4050, 2000; UC199900546bEN NP-3678EN, 2000. Medtronic, Inc., Minneapolis, Minn. (1997-2000). Many pumps have 2 ports: one into which drugs can be injected and the other that is connected directly to the catheter for bolus administration or analysis of fluid from the catheter. Implantable drug infusion pumps (e.g., SynchroMed EL and SynchroMed programmable pumps; Medtronic) are indicated for long-term intrathecal infusion of morphine sulfate for the treatment of chronic intractable pain; intravascular infusion of floxuridine for treatment of primary or metastatic cancer; intrathecal injection (baclofen injection) for severe spasticity; long-term epidural infusion of morphine sulfate for treatment of chronic intractable pain; long-term intravascular infusion of doxorubicin, cisplatin, or methotrexate for the treatment or metastatic cancer; and long-term intravenous infusion of clindamycin for the treatment of osteomyelitis. Such pumps may also be used for the long-term infusion of one or more compounds simultaneously, including, a dithio-containing compound of the present invention, in combination with one or more chemotherapeutic agents of choice, at a desired amount for a desired number of doses or steady state administration. One form of a typical implantable drug infusion pump (e.g., SynchroMed EL programmable pump; Medtronic) is titanium covered and roughly disk shaped, measures 85.2 mm in diameter and 22.86 mm in thickness, weighs 185 g, has a drug reservoir of 10 mL, and runs on a lithium thionyl-chloride battery with a 6- to 7-year life, depending on use. The downloadable memory contains programmed drug delivery parameters and calculated amount of drug remaining, which can be compared with actual amount of drug remaining to access accuracy of pump function, but actual pump function over time is not recorded. The pump is usually implanted in the right or left abdominal wall. Other pumps useful in the present invention include, for example, Portable Disposable Infuser Pumps (PDIPs). Additionally, implantable infusion devices may employ liposome delivery systems, such as a small unilamellar vesicles, large unilamellar vesicles, and multilamellar vesicles that can be formed from a variety of phospholipids, such as cholesterol, stearyl amine, or phosphatidylcholines.

The present invention also provides in part dose delivery formulations and devices formulated to enhance bioavailability of a dithio-containing compound of the present invention. This may be in addition to or in combination with one or more chemotherapeutic agents, or any of the formulations and/or devices described above.

For example, an increase in bioavailability of a dithio-containing compound of the present invention, may be achieved by complexation of a dithio-containing compound of the present invention, with one or more bioavailability or absorption enhancing agents or formulations, including bile acids such as taurocholic acid.

The present invention also provides for the formulation of a dithio-containing compound of the present invention, as well as one or more chemotherapeutic agents, in a microemulsion to enhance bioavailability. A microemulsion is a fluid and stable homogeneous solution composed of four major constituents, respectively, a hydrophilic phase, a lipophilic phase, at least one surfactant (SA) and at least one cosurfactant (CoSA). A surfactant is a chemical compound possessing two groups, the first polar or ionic, which has a great affinity for water, the second which contains a longer or shorter aliphatic chain and is hydrophobic. These chemical compounds having marked hydrophilic character are intended to cause the formation of micelles in aqueous or oily solution. Examples of suitable surfactants include mono-, di- and triglycerides and polyethylene glycol (PEG) mono- and diesters. A cosurfactant, also sometimes known as “co-surface-active agent”, is a chemical compound having hydrophobic character, intended to cause the mutual solubilization of the aqueous and oily phases in a microemulsion. Examples of suitable co-surfactants include ethyl diglycol, lauric esters of propylene glycol, oleic esters of polyglycerol, and related compounds.

Any such dose may be administered by any of the routes or in any of the forms herein described. For example, a dose or doses could be given parenterally using a dosage form suitable for parenteral administration which may incorporate features or compositions described in respect of dosage forms delivered in a modified release, extended release, delayed release, slow release or repeat action oral dosage form.

The present invention also provides for the formulation of a dithio-containing compound of the present invention, for rectal delivery and absorption via the utilization of rectal suppositories or retention enemas. Generally, suppositories are utilized for delivery of drugs to the rectum and sigmoid colon. The ideal suppository base for the delivery of the formulations of the present invention should meet the following specifications: (i) a base which is non-toxic and non-irritating to the anal mucous membranes; (ii) a base which is compatible with a variety of drugs; (iii) a base which melts or dissolves in rectal fluids; and (iv) a base which is stable in storage and does not bind or otherwise interfere with the release and/or absorption of the pharmaceutical formulations contained therein. Typical suppository bases include: cocoa butter, glycerinated gelatine, hydrogenated vegetable oils, mixtures of polyethylene glycols of various molecular weights and fatty acid esters of polyethylene glycol. The rectal Epithelium is lipoidal in character. The lower, middle, and upper hemorrhoidal veins surrounds the rectum. Only the upper vein conveys blood into the portal system, thus drugs absorbed into the lower and middle hemorrhoidal veins will bypass the liver and the cytochrome P₄₅₀ oxidase system. Absorption and distribution of a drug is therefore modified by its position within the rectum, in that at least a portion of the drug absorbed from the rectum may pass directly into the inferior vena cava, bypassing the liver. The present invention also provides for the formulation of a dithio-containing compound of the present invention, as well as one or more chemotherapeutic agents, administered by suppository.

A better understanding of the invention will be gained by reference to the following specific examples. The following examples are illustrative and are not intended to limit the invention or the claims in any way.

### SPECIFIC EXAMPLES OF EXPERIMENTAL RESULTS

### I. Effects of Tavocept™ on Glutathione-S-Transferase (GST)

One potential hypothesis set forth to explain the ability of Tavocept™ (disodium 2,2′-dithio-bis-ethane sulfonate) to augment the anti-cancer activity of chemotherapeutic agents states that Tavocept™ may act as a glutathione surrogate or modulator in the reactions of glutathione-S-transferase (GST). Glutathione and its related enzymes play a major role in the detoxification of toxic chemicals including cytotoxic chemotherapeutics. Glutathione-S-transferases (GSTs) constitute a family of phase II detoxifying isozymes that catalyze the conjugation of glutathione to a variety of electrophilic compounds, often the first step in the formation of mercapturic acid derivatives such as N-acetylcysteine. Reaction Scheme I, below, illustrates Glutathione S-transferase catalyzing the transfer of glutathione to an electrophilic species RX (wherein, R is S, N or C).

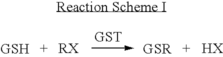

The resulting glutathione conjugates are either excreted from the cell or they undergo further enzymatic processing by γ-glutamyl transpeptidase and cysteine-S-conjugate-β-lyase. See, e.g., Hausheer, F. H., et al., Modulation of platinum-induced toxicities and therapeutic index: mechanistic insights and first- and second-generation protecting agents. Semin Oncol. 25:584-599 (1998). Glutathione-S-transferases (GSTs) are highly expressed in tumor tissue relative to normal tissues and are also found in high levels in the plasma of cancer patients; thereby making these enzymes useful as potential cancer markers. There are multiple cytosolic- and membrane-bound GST isozymes that differ in their tissue-specific expression and distribution. GSTs protect mammalian cells against the toxic and neoplastic effects of electrophilic metabolites of carcinogens and reactive oxygen species. For example, increased expression of GSTs has been linked to the development of cellular resistance to alkylating cytostatic drugs. A deficiency of GST isozymes may increase the predisposition to various forms of cancer. Therefore, GST status may be a useful diagnostic factor in determining the clinical outcome of chemotherapy.

The following experiments were designed to determine if Tavocept™ has an inhibitory or stimulatory effect on GST. Specifically, these studies address whether Tavocept™ can act as a substrate for GST or if either of these compounds inhibit GST. An in vitro assay for GST has been developed and reported. See, Meyer, D. J. and Ketterer, B., Purification of soluble human glutathione S-transferases. Methods Enzymol. 252:53-65 (1995). This assay monitors the conjugation of reduced glutathione to 1-chloro-2,4-dinitrobenzene (CDNB), as illustrated in Reaction Scheme II, below.

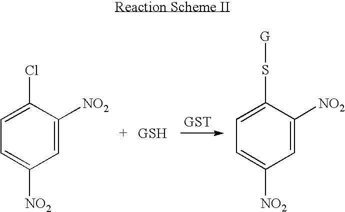

Reduced thiol forms a conjugate with CDNB (extinction coefficient=9600 M⁻¹ cm⁻¹), which is detected at 340 nm. Stock solutions of GSH, CDNB, Tavocept™ were prepared by dissolving the reagent in sterile water at the concentrations listed below prior to use. A typical 1 mL assay was set up by mixing 500 AL NaHPO₄ buffer (200 mM, pH 6.5), 20 μL GSH (50 mM), 20 μL CDNB (50 mM), and 458 μL sterile water. Reactions were incubated at 20° C. in the cuvette holder of the spectrophotometer for approximately 5 min. prior to initiating the assay with the addition of enzyme (m1-1 isotype of GST; activity >100 U/mg). The enzyme stock purchased from the vendor was diluted 1:100 in 200 mM NaHPO₄ buffer (pH 6.5), and 2 μL of the diluted enzyme was added to initiate the reaction. The final amount of enzyme added to the assay was typically 0.002 U. Assays were run at 20° C. in 1 mL quartz cuvettes (Hellma Scientific). Slopes were measured in the linear range of the assay (i.e., typically between 5 to 10 min.). In assays where the effect of Tavocept™ on GST activity was measured, 20 μL of either a 500 mM, 166.7 mM, or 55.6 mM stock solution of Tavocept™ was added to standard reactions using 1 mM GSH as the enzyme substrate. Final reaction volumes were fixed at 1 mL by adjusting the amount of water added.

All UV-visible assays were performed using a Varian Cary 100 spectrophotometer equipped with a thermostatic jacketed multi-cell holder. The default parameters of the Cary Win UV Enzyme Kinetics application (version 2.00) were used; with the exceptions of using both the visible and deuterium lamps, and setting the wavelength to 340 nm, the temperature to 20° C., and the maximal duration of the assay at 30 minutes.

Raw data was obtained on a Cary 100 spectrophotometer. This data showed several phases to a typical reaction. The first phase was a baseline corresponding to the time prior to addition of enzyme (typically 2-5 min. in duration). Assays in the first phase of the reaction contained only substrate, buffer and (in some assays) Tavocept™. The spectrophotometer was put in pause mode while enzyme (GST) was added and mixed into the assay reactions. No absorbance values were collected during the process of enzyme addition. The region of experimental interest was during the linear phase of the enzyme reaction, which immediately followed the addition of enzyme. The linear phase is of experimental interest because it is when the classical model of Michaelis-Menton kinetics holds true. During this phase the substrate concentration is high (>Km for enzyme) and, therefore, the rate of catalysis is independent of the substrate concentration. It was during this time that reaction rates (i.e., slopes of change in absorbance with time) were measured using the Cary 100 software. The duration of the linear phase was between 5-10 minutes, depending upon the specific reaction conditions. Reactions were considered complete when substrate concentration was no longer saturating and became a rate limiting factor of the assay. When the substrate was limiting, the reaction rate deviated from linearity. This end phase of the reaction was typically observed after 10 to 15 minutes. Absorbance and time values during the end phase of the reaction were not used in slope calculations because the reaction was effectively over at this point as the reaction no longer followed the classical Michaelis-Menton model for enzyme kinetics. Completion of the reaction on the Cary software could be detected visually by overlaying a straight line beginning at the addition of enzyme and extending past the end phase of the assay curve. Upon completion of a set of reactions data was stored as an electronic “batch” file. Sigma Plot was used. specifically to show the mean of assays run in triplicate with linear regression lines and error bars illustrating standard deviation. Descriptive statistics (mean and standard deviation) were used to describe and summarize the results of the experiments. The results of these experiments are illustrated in Graph I. below.

The GST reaction was performed in the presence of Tavocept™. Final Tavocept™ concentrations are shown to the right of each regression curve. Data points shown represents the average curve of triplicate experiments for each assay condition, and error bars are standard deviation. Assays were measured after the addition of GST in the linear range (i.e., 8.9 min. to 13.1 min.).

The individual slopes for each of the three assay runs for a given Tavocept™ concentration, the standard deviation, the mean, the relative enzyme activity, and percent inhibition are listed in Table 2, below.

Table 2 shows the slopes for each assay trial, which were calculated from the change in absorbance at 340 nm per minute in the linear portion of the assay. In these examples, the slope was measured from 8.9 to 13.1 min. The relative activity was normalized using the slope mean to the reactions having no Tavocept™ added; and percent inhibition was calculated as the difference of relative activity from 100%.

TABLE 2
Rates of GST Assays Run in the Presence of Tavocept ™

| Tavocept ™ | Slope | Standard | Slope | Relative | Percent |
|---|---|---|---|---|---|
| Concentration | Abs/min | Deviation | Mean | Activity | Inhibition |
| 0 mM | 0.0465 | 0.0029 | 0.0449 | 100% | 0 |
| 0 mM | 0.0424 | 0.0023 | | | |
| 0 mM | 0.0458 | 0.0023 | | | |
| 1.1 mM | 0.0427 | 0.0023 | 0.0424 | 94.4% | 5.6 |
| 1.1 mM | 0.0437 | 0.0020 | | | |
| 1.1 mM | 0.0407 | 0.0020 | | | |
| 3.3 mM | 0.0295 | 0.0014 | 0.0274 | 61% | 39 |
| 3.3 mM | 0.0242 | 0.0009 | | | |
| 3.3 mM | 0.0284 | 0.0011 | | | |
| 10 mM | 0.0155 | 0.0012 | 0.0151 | 33.6% | 66.4 |
| 10 mM | 0.0158 | 0.0012 | | | |
| 10 mM | 0.0139 | 0.0009 | | | |

Accordingly, the data obtained from both Graph I and Table 2 illustrate that increased concentrations of Tavocept™ cause a marked increase in the percent of inhibition of GST catalysis in the conjugation of reduced glutathione to 1-chloro-2,4-dinitrobenzene (CDNB), as initially illustrated in Reaction Scheme II, above. For example, an increase of Tavocept™ from 1.1 mM to 3.3 mM was shown to cause an increase in the percent inhibition from 5.6% to 39.0%. Thus, this relatively small increase in Tavocept™ concentration caused an approximate 6-times increase in GST inhibition.

One function of GST and related species (GSTs) is to protect mammalian cells against the neoplastic effects of electrophilic metabolites of carcinogens and reactive oxygen species by, e.g., catalyzing the conjugation of glutathione to a variety of electrophilic compounds. Moreover, GSTs are highly expressed in tumor tissue relative to normal tissues, are found in high levels in the plasma of cancer patients, and increased expression of GSTs has been linked to the development of cellular resistance to alkylating cytostatic drugs. Thus, it is probable that one possible mechanism of action of Tavocept™ may be to cause a change or changes in the intracellular oxidative/reductive potential within tumor cells so as to increase intracellular oxidative stress. This change may, in turn, cause the tumor cell to exhibit greater sensitivity to a chemotherapeutic agent without directly affecting the mechanism of action of the chemotherapeutic agent itself. By way of non-limiting example, this increased sensitivity would allow: (i) increased anti-tumor effects for a given chemotherapeutic dosage; (ii) decrease in the administered chemotherapeutic dose; (iii) decrease in the overall length of the chemotherapeutic cycle; and (iv) decrease in the length of time between courses of chemotherapy.

### II. Tavocept as Substrate for Thioredoxin (TRX)

The TRX system plays an important role in the redox regulation of a number of cellular processes, notably modulation of apoptosis and cellular proliferation. The system includes the selenoprotein, thioredoxin reductase (TRR), and its main substrate, thioredoxin (TRX), as well as thioredoxin peroxidase (TPX). See, e.g., Zhong, L., et al., Rat and calf thioredoxin reductase are homologous to glutathione reductase with a carboxyl-terminal elongation containing a conserved catalytically active penultimate seloncysteine residue. J. Biol. Chem. 273: 8581-8591, 1998 Holmgren, A. Thioredoxin and glutaredoxin systems. J. Biol. Chem. 264:13963-13966 (1989). TRR is a pyridine nucleotide-disulfide oxidoreductase, and catalyzes the NADPH-dependent reduction of the active site disulfide in oxidized thioredoxin (see, Reaction Scheme III; TRX-S₂) to give a dithiol in reduced thioredoxin (TRX-(SH)₂). See, e.g., Zhong, L., et al. Rat and calf thioredoxin reductase are homologous to glutathione reductase with a carboxyl-terminal elongation containing a conserved catalytically active penultimate seloncysteine residue. J. Biol. Chem. 273:8581-8591 (1998). Reaction Scheme III, below, outlines the various reaction mechanisms involved in the TRX redox regulation system.

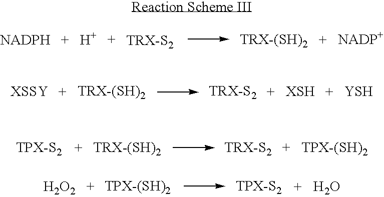

TRX is a small disulfide reductase with a broad range of substrates and important functions in the redox modulation of protein signaling and the reductive activation of a number of important transcription factors. See, e.g., Welsh, S. J., et al., The thioredoxin redox inhibitors 1-methylpropyl 2-imidazolyl disulfide and pleurotin inhibit hypoxiainduced factor 1alpha and vascular endothelial growth factor formation. Mol. Cancer Therapy 2:235-243 (2003). Like GRX, TRX is only active in its reduced form (TRX-(SH)₂) which serves as a hydrogen donor for ribonucleotide reductase and other redox enzymes, and acts in defense against oxidative stress. While they share some substrate specificity, the TRX system is more catalytically diverse than the GRX system and does not interact substantially with glutathione. See, e.g., Luthman, M., and Holmgren, A. Rat liver thioredoxin and thioredoxin reductase: purification and characterization. Biochemistry 21:6628-6633 (1982).

The objective of this study was to determine if Tavocept™ has a detectable, direct interaction with the following oxidoreductase enzymes: glutathione reductase (GR); glutaredoxin (GRX); glutathione peroxidase (GPX); thioredoxin reductase (TRR); and thioredoxin (TRX). Based upon the nature and magnitude of the interaction, it may be determined whether an interaction with redox balance enzymes could serve to explain clinical findings regarding Tavocept™ metabolism or its mechanism of action.

The activity of TRR and TRX was determined by following NADPH oxidation at 340 nm according to the previously reported method. See, Luthman, M., and Holmgren, A. Rat liver thioredoxin and thioredoxin reductase: purification and characterization. Biochemistry 21:6628-6633 (1982). A typical assay mixture contained TR buffer (50 mM potassium phosphate, pH 7.0, 1 mM EDTA), 200 μM NADPH, 1.6 μg bovine TRR, and one or more of the following: 4.8 μM TRX, 86 μM insulin, and one of the disulfides described herein. All disulfides were added to reactions as 10× solutions in TR buffer. The total volume of each reaction was 0.1 mL. Reactions were initiated by the addition of TRR and were incubated at 25° C. for 40 min. The activity was calculated using a 4 min. linear portion of each reaction. Enzyme assays were carried out using either a Molecular Devices SpectraMaxPlus UV plate reader or a Varian Cary 100 UV-visible Spectrophotometer.

Data was then collected and plotted in Microsoft Excel. Error calculations, and graphical representations were performed in Microsoft Excel and Kaleidograph (ver. 3.5). Nonlinear data was graphically rendered using Kaleidograph. ANOVA and other statistical analyses were performed using SAS (ver. 8.2). Unless otherwise noted, significance level was set at 0.05, and error bars represent actual experimental standard deviation.

The activity of TRR and TRX with Tavocept™ is depicted in Graph III, below. Tavocept™ causes a concentration-dependent increase in NADPH oxidation by TRR in the presence of TRX. In the absence of TRX, the NADPH oxidation by TRR is indistinguishable from background. Based upon the magnitude and concentration-dependence of the observed oxidation responses, Tavocept™ is most likely a substrate for TRX, but not for TRR.

Mechanisms of Action of Tavocept

An important element of Tavocept's™ effectiveness as a compound in the treatment of cancer is its selectivity for normal cells versus cancer cells and its inability to interfere with the anti-cancer activity of chemotherapeutic agents. In vitro studies demonstrated that Tavocept™ does not interfere with paclitaxel induced apoptosis, as assessed by PARP cleavage, Bcl-2 phosphorylation, and DNA laddering in human breast, ovarian and lymphoma cancer cell lines. Additionally, Tavocept™ did not interfere with paclitaxel and platinum induced cytotoxicity in human cancer cell lines and did not interfere with paclitaxel and platinum regimens in the animals models discussed herein.

The potential mechanisms underlying the absence of interference with anti-cancer activity by Tavocept™ are multifactorial and, as previously discussed, may involve its selectivity for normal cells versus cancer cells, inherent chemical properties that have minimal impact on critical plasma and cellular thiol-disulfide balances, and its interactions with cellular oxidoreductases, which are key in the cellular oxidative/reduction (redox) maintenance systems.

In addition to the absence of interference with anti-cancer activity, results from in vivo studies have shown that Tavocept™ may elicit the restoration of apoptotic sensitivity in tumor cells through thioredoxin- and glutaredoxin-mediated mechanisms and this may be an important element of its effectiveness as a chemotherapeutic agent. It has been determined that Tavocept™ is a substrate for thioredoxin and exhibits substrate-like activity with glutaredoxin in the presence of reduced glutathione and glutathione reductase, and this substrate-like activity may be due to non-enzymatic formation of glutathione-containing disulfide heteroconjugates during the assay reaction; these glutathione disulfide heteroconjugates may, in turn, act as substrates for glutaredoxin. Thus, Tavocept™ could potentially shift the intracellular balance of oxidized (inactive) and reduced (active) thioredoxin or glutaredoxin, subsequently modulating their cellular activity.

Similarly, as previously shown, increased concentrations of Tavocept™ cause a marked increase in the percent of inhibition of GST catalysis in the conjugation of reduced glutathione to 1-chloro-2,4-dinitrobenzene (CDNB). One function of GST and related species (GSTs) is to protect mammalian cells against the neoplastic effects of electrophilic metabolites of carcinogens and reactive oxygen species by, e.g., catalyzing the conjugation of glutathione to a variety of electrophilic compounds. Moreover, GSTs are highly expressed in tumor tissue relative to normal tissue, are found in high levels in the plasma of cancer patients, and increased expression of GSTs has been linked to the development of cellular resistance to alkylating cytostatic drugs.

Tavocept™ restoration of the apoptotic sensitivity of tumor cells via thioredoxin, glutaredoxin or related cellular redox systems, would have a net anti-proliferative activity on tumor cells. Thioredoxin and GST are key players both in apoptotic pathways in cells and in the intracellular redox environment and any molecule that inhibits or serves as substrate for these proteins could offset changes in the intracellular redox environments that are due to high/elevated/aberrant levels of thioredoxin and/or GST. The effect of Tavocept™ on thioredoxin and/or GST could also potentially normalize redox sensitive signaling pathways that are involved in apoptosis. Thus, the net results would be an increased sensitivity of tumor cells to chemotherapeutic agents and/or restoration of a more normal intracellular redox environment A substantial increase in the inactive forms of these oxidoreductases could result in significant changes in redox homeostasis, cell proliferation, and gene transcription through reductive control over various transcription factors. Specifically, the involvement of the thioredoxin system in tumor progression, its influence on p53-mediated gene transcription, and its demonstrated roles in neuroprotection against chemical toxins would indicate that interaction of this system with Tavocept™ could have a variety of positive clinical sequelae including: (i) inhibition of tumor growth in the presence of oxidative stressors; (ii) protection of normal cells during chemically-induced hyperoxidation and hyperthermia of cancer cells; and/or (iii) amelioration of chemically-induced neurotoxicity.

In conclusion, the Applicant believes the data discussed above supports the ability of Tavocept™ to augment the anti-cancer activity of chemotherapeutic agents by increasing oxidative stress within tumor cells (i.e., by physiological and pharmacological thiol depletion, thioredoxin inactivation, increasing the oxidative biological state and/or associated oxidative damage within said tumor cells, thereby enhancing the cytotoxicity and apoptotic ability of chemotherapeutic agents), in a selective manner, while avoiding deleterious chemotherapeutic agent-induced effects on normal (i.e., non-cancerous) cells and tissues. As previously discussed, Tavocept™ (disodium 2,2′-dithio-bis ethane sulfonate) has been introduced into Phase I, Phase II, and Phase III clinical testing in patients, as well as in non-clinical testing, by the Assignee of the of the present invention, BioNumerik Pharmaceuticals, Inc., under the guidance of the Applicant. The Applicant believes that further evaluation of the data from this testing will lend further support for the ability of Tavocept™ to augment the anti-cancer activity of chemotherapeutic agents as disclosed in the present invention.

All patents, publications, scientific articles, web sites, and the like, as well as other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicant reserves the right to physically incorporate into this specification any and all materials and information from any such patents, publications, scientific articles, web sites, electronically available information, and other referenced materials or documents.

The written description portion of this patent includes all claims. Furthermore, all claims, including all original claims as well as all claims from any and all priority documents, are hereby incorporated by reference in their entirety into the written description portion of the specification, and Applicant reserves the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in the written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, the terms “comprising”, “including”, “containing”, etc. are to be read expansively and without limitation. The methods and processes illustratively described herein suitably may be practiced in differing orders of steps, and they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various embodiments and/or preferred embodiments and optional features, any and all modifications and variations of the concepts herein disclosed that may be resorted to by those skilled in the art are considered to be within the scope of this invention as defined by the appended claims.

The present invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”. The letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and Applicant reserves the right to revise the application or claims to refer specifically to any individual member or any subgroup of members of the Markush group.

Other embodiments are within the following claims. The patent may not be interpreted to be limited to the specific examples or embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

What is claimed is:

1. A method of augmenting the anti-cancer cytotoxic activity of chemotherapeutic agents by acting in an additive or synergistic cytotoxic manner with said chemotherapeutic agent or agents to increase intracellular oxidative stress within cancer cells, wherein said method comprises administering to a subject who has received one or more chemotherapeutic agents an effective amount of a pharmaceutically-acceptable form of a dithio-containing compound of the present invention at a rate of about 0.1 g/min. to about 2.0 g/min.

2. The method of claim 1, wherein said dithio-containing compound of the present invention is administered at a rate of about 0.2 g/min. to about 1.0 g/min.

3. The method of claim 1, wherein said dithio-containing compound of the present invention is administered at a rate of about 0.7 g/min.

4. The method of any one of claims 1, 2 or 3, wherein said dithio-containing compound is administered over a period of about 45 minutes.

5. The method of any one of claims 1, 2 or 3, wherein said dithio-containing compound of the present invention is administered at a concentration of about 100 mg/mL.

6. The method of any one of claims 1, 2 or 3, wherein said dithio-containing compound of the present invention is administered over a period of about 45 minutes, at a concentration of about 100 mg/mL.

7. The method of any one of claims 1, 2 or 3, wherein said dithio-containing compound of the present invention is administered from about once a day to about once every five weeks.

8. The method of claim 1, wherein said dithio-containing compound of the present invention is administered from about once a day to about once every three weeks.

9. The method of claim 1, wherein said dithio-containing compound of the present invention is administered from about once a day to about once every two weeks.

10. The method of claim 1, wherein said dithio-containing compound of the present invention is administered from about once a day to about once every week.

11. The method of claim 1, wherein said dithio-containing compound of the present invention is administered in a time period which is selected from the group consisting of: about once every day, about once every two days, about once every three days, about once every four days, once about every five days, or about once every six days.

12. The method of any one of claims 1, 2, or 3, wherein said dithio-containing compound of the present invention is administered in a time period which is selected from the group consisting of: at least once in an approximately 24 hour period; at least once in an approximately 48 hour period; at least about once every three days; at least about once every four days; at least about once every five days; at least about once every six days; at least about once a week; at least about once every 1.5 weeks or less; at least about once every 2 weeks or less; at least about once every 2.5 weeks or less; at least about once every 3 weeks or less; at least about once every 3.5 weeks or less; at least about once every 4 weeks or less; at least about once every 5 weeks or less; at least once at any time interval between one day and five weeks; or at least once at a time interval of more than every five weeks.

13. The method of claim 1, wherein the augmentation of the anti-cancer activity of said chemotherapeutic agent or agents in said subject who has received one or more chemotherapeutic agents, and an effective amount of a dithio-containing compound of the present invention is caused by a prevention and/or reduction in the normal increase or responsiveness in the concentration and metabolism of glutathione, cysteine, and other physiological cellular thiols.

14. The method of claim 1, wherein the augmentation of the anti-cancer activity of said chemotherapeutic agent or agents in said subject who has received one or more chemotherapeutic agents, and an effective amount of a dithio-containing compound of the present invention is caused by thioredoxin inactivation, thereby increasing apoptotic sensitivity and decreasing mitogenic/cellular replication signaling.

15. The method of claim 1, wherein the augmentation of the anti-cancer activity of said chemotherapeutic agent or agents in said subject who has received one or more chemotherapeutic agents, and an effective amount of a dithio-containing compound of the present invention is caused by a key metabolite of said dithio-containing compound, such as 2-mercapto ethane sulfonate, which possesses intrinsic cytotoxic activity and causes apoptosis in tumors.

16. The method of claim 1, wherein the augmentation of the anti-cancer activity of said chemotherapeutic agent or agents in said subject who has received one or more chemotherapeutic agents, and an effective amount of a dithio-containing compound of the present invention is caused by reducing the oxidative potential or by compromising the anti-oxidative response of tumor cells and enhancing the oxidative biological state and oxidative damage in tumor cells exposed to chemotherapeutic agents, thereby enhancing the cytotoxic and apoptotic function of said chemotherapeutic agents.

17. The method of claim 1, wherein said chemotherapeutic agent or agents are selected from a group consisting of: fluropyrimidine; a pyrimidine nucleoside; a purine nucleoside; an antifolate, a platinum analog; an anthracycline/anthracenedione; an epipodophyllotoxin; a camptothecin; a hormone; a hormonal analog; an antihormonal; an enzyme, protein, peptide, or polyclonal or monoclonal antibody; a vinca alkaloid; a taxane; an epothilone; an antimicrotubule agent; an alkylating agent; an antimetabolite; a topoisomerase inhibitor; an aziridine-containing compound; an antiviral; or another cytotoxic and/or cytostatic agent.

18. The method of claim 1, wherein the chemotherapeutic agent is a taxane analog.

19. The method of claim 1, wherein the chemotherapeutic agent is docetaxel.

20. The method of claim 1, wherein the chemotherapeutic agent is paclitaxel.

21. The method of claim 1, wherein the chemotherapeutic agent is a platinum analog.

22. The method of claim 1, wherein the chemotherapeutic agent is cisplatin.

23. The method of claim 1, wherein the chemotherapeutic agent is carboplatin.

24. The method of claim 1, wherein the chemotherapeutic agent is oxaliplatin.

25. The method of claim 1, wherein said method is utilized for the treatment of a subject with cancer.

26. The method of claim 25, wherein said subject is human.

27. The method of claim 25 or claim 26, wherein said method is utilized for treating any one or more cancers selected from the group consisting of: ovary, breast, lung, esophagus, bladder, stomach, pancreas, liver (e.g., bile ducts, gall bladder, and Ampulla of Vater), testes, germ cell, bone, cartilage, head, neck, oral mucosa, colorectal area, anus, kidney, uroepithelium, central nervous system, prostate, endometrium, cervix, uterus, fallopian tube, peripheral nervous system, and various other cancers including melanoma, mesothelioma, myeloma, lymphoma, leukemia, and Kaposi's sarcoma.

28. The method of claim 1, wherein said method further comprises the prevention, reduction or mitigation of one or more toxicities associated with administration of said chemotherapeutic agent or agents.

29. The method of claim 1, wherein the augmentation of the anti-cancer activity of the chemotherapeutic agent allows said chemotherapeutic agent to be administered at a lower dose, while still achieving the same degree of clinical efficacy as would be obtained with the administration of a higher dose.

30. The method of claim 1, wherein said dithio-containing compound is disodium 2,2′-dithio-bis-ethane sulfonate.

31. The method of claim 1, wherein said dithio-containing compound of the present invention is a pharmaceutically-acceptable salt.

32. The method of claim 31, wherein said salt is a disodium salt.

33. The method of claim 31, wherein said salt is selected from the group consisting of: a monosodium salt, a sodium potassium salt, a dipotassium salt, a calcium salt, a magnesium salt, a manganese salt, a monopotassium salt, or an ammonium salt.

34. The method claim 1, further comprising administering a pre-therapy treatment at least one day prior to chemotherapy, prior to each chemotherapy treatment, immediately prior to each chemotherapy treatment, concomitantly with or simultaneously during chemotherapy treatment, immediately subsequent to chemotherapy, or subsequent to chemotherapy.

35. The method of claim 1, further comprising a hydration step.

36. The method of any one of claims 1, 2, or 3, wherein said dithio-containing compound of the present invention is in a form suitable for administration by a method selected from the group consisting of: oral, injection, intra-cavitary, per rectum, and topical administration routes.

37. A method of augmenting the anti-cancer cytotoxic activity of chemotherapeutic agents by acting in an additive or synergistic cytotoxic manner with said chemotherapeutic agent or agents to increase intracellular oxidative stress within cancer cells, wherein said method comprises administering to a subject who has received one or more chemotherapeutic agents an effective amount of a pharmaceutically-acceptable form of a dithio-containing compound of the present invention at a rate of about 1 mg/mL/min. to about 50 mg/mL/min.

38. The method of claim 37, wherein said dithio-containing compound of the present invention is administered at a rate of about 1 mg/mL/min. to about 20 mg/mL/min.

39. The method of claim 37, wherein said dithio-containing compound of the present invention is administered at a rate of about 7 mg/mL/min.

40. The method of any one of claims 37, 38, or 39, wherein said dithio-containing compound of the present invention is administered over a period of about 45 minutes.

41. The method of any one of claims 37, 38, or 39, wherein said dithio-containing compound of the present invention is administered at a concentration of about 100 mg/mL.

42. The method of any one of claims 37, 38, or 39, wherein said dithio-containing compound of the present invention is administered over a period of about 45 minutes, at a concentration of about 100 mg/mL.

43. The method of any one of claims 37, 38, or 39, wherein said dithio-containing compound of the present invention is administered from about once a day to about once every five weeks.

44. The method of claim 37, wherein said dithio-containing compound of the present invention is administered from about once a day to about once every three weeks.

45. The method of claim 37, wherein said dithio-containing compound of the present invention is administered from about once a day to about once every two weeks.

46. The method of claim 37, wherein said dithio-containing compound of the present invention is administered from about once a day to about once every week.

47. The method of claim 37, wherein said dithio-containing compound of the present invention is administered in a time period which is selected from the group consisting of: about once every day, about once every two days, about once every three days, about once every four days, once about every five days, or about once every six days.

48. The method of any one of claim 37, 38, or 39, wherein said dithio-containing compound of the present invention is administered in a time period which is selected from the group consisting of: at least once in an approximately 24 hour period; at least once in an approximately 48 hour period; at least about once every three days; at least about once every four days; at least about once every five days; at least about once every six days; at least about once a week; at least about once every 1.5 weeks or less; at least about once every 2 weeks or less; at least about once every 2.5 weeks or less; at least about once every 3 weeks or less; at least about once every 3.5 weeks or less; at least about once every 4 weeks or less; at least about once every 5 weeks or less; at least once at any time interval between one day and five weeks; or at least once at a time interval of more than every five weeks.

49. The method of claim 37, wherein the augmentation of the anti-cancer activity of said chemotherapeutic agent or agents in said subject who has received one or more chemotherapeutic agents, and an effective amount of a dithio-containing compound of the present invention is caused by a prevention and/or reduction in the normal increase or responsiveness in the concentration and metabolism of glutathione, cysteine, and other physiological cellular thiols.

50. The method of claim 37, wherein the augmentation of the anti-cancer activity of said chemotherapeutic agent or agents in said subject who has received one or more chemotherapeutic agents, and an effective amount of a dithio-containing compound of the present invention is caused by thioredoxin inactivation, thereby increasing apoptotic sensitivity and decreasing mitogenic/cellular replication signaling.

51. The method of claim 37, wherein the augmentation of the anti-cancer activity of said chemotherapeutic agent or agents in said subject who has received one or more chemotherapeutic agents, and an effective amount of a dithio-containing compound of the present invention, e.g., 2,2′-dithio-bis-ethane sulfonate is caused by a key metabolite of 2,2′-dithio-bis-ethane sulfonate, 2-mercapto ethane sulfonate, which possesses intrinsic cytotoxic activity and causes apoptosis in tumors.

52. The method of claim 37, wherein the augmentation of the anti-cancer activity of said chemotherapeutic agent or agents in said subject who has received one or more chemotherapeutic agents, and an effective amount of a dithio-containing compound of the present invention is caused by reducing the oxidative potential or by compromising the anti-oxidative response of tumor cells and enhancing the oxidative biological state and oxidative damage in tumor cells exposed to chemotherapeutic agents, thereby enhancing the cytotoxic and apoptotic function of said chemotherapeutic agents.

53. The method of claim 37, wherein said chemotherapeutic agent or agents are selected from a group consisting of: fluropyrimidines, pyrimidine nucleosides, anti-folates, purine nucleosides, nucleotides, platinum analogs, anthracyclines/anthracenediones, epipodophyllotoxins, camptothecins, hormones and hormonal analogs, antihormones, enzymes, proteins, antibodies, vinca alkaloids, taxanes and taxane analogs, antimicrotubule agents, alkylating agents, epothilones, antimetabolites, topoisomerase inhibitors, aziridine-containing compounds, antiviral agents, monoclonal antibodies, proteins, peptides, enzymes, or cytostatic agents.

54. The method of claim 37, wherein the chemotherapeutic agent is a taxane analog.

55. The method of claim 37, wherein the chemotherapeutic agent is docetaxel.

56. The method of claim 37, wherein the chemotherapeutic agent is paclitaxel.

57. The method of claim 37, wherein the chemotherapeutic agent is a platinum analog.

58. The method of claim 37, wherein the chemotherapeutic agent is cisplatin.

59. The method of claim 37, wherein the chemotherapeutic agent is carboplatin.

60. The method of claim 37, wherein the chemotherapeutic agent is oxaliplatin.

61. The method of claim 37, wherein said method is utilized for the treatment of a subject with cancer.

62. The method of claim 61, wherein said subject is human.

63. The method of claim 61 or claim 62, wherein said method is utilized for treating any one or more cancers selected from the group consisting of: ovary, breast, lung, esophagus, bladder, stomach, pancreas, liver (e.g., bile ducts, gall bladder, and Ampulla of Vater), testes, germ cell, bone, cartilage, head, neck, oral mucosa, colorectal area, anus, kidney, uroepithelium, central nervous system, prostate, endometrium, cervix, uterus, fallopian tube, peripheral nervous system, and various other cancers including melanoma, mesothelioma, myeloma, lymphoma, leukemia, and Kaposi's sarcoma.

64. The method of any one of claim 37, 38, or 39, wherein said method further comprises the prevention, reduction or mitigation of one or more toxicities associated with administration of said chemotherapeutic agent or agents.

65. The method of any one of claim 37, 38, or 39, wherein the augmentation of the anti-cancer activity of the chemotherapeutic agent allows said chemotherapeutic agent to be administered at a lower dose, while still achieving the same degree of clinical efficacy as would be obtained with the administration of a higher dose.

66. The method of any one of claim 37, 38, or 39, wherein said dithio-containing compound is disodium 2,2′-dithio-bis-ethane sulfonate.

67. The method of claim 37, wherein said dithio-containing compound of the present invention is a pharmaceutically-acceptable salt.

68. The method of claim 67, wherein said salt is a disodium salt.

69. The method of claim 67, wherein said salt is selected from the group consisting of: a monosodium salt, a sodium potassium salt, a dipotassium salt, a calcium salt, a magnesium salt, a manganese salt, a monopotassium salt, or an ammonium salt.

70. The method of claim 37, further comprising administering a pre-therapy treatment at least one day prior to chemotherapy, prior to each chemotherapy treatment, immediately prior to each chemotherapy treatment, concomitantly with or simultaneously during chemotherapy treatment, immediately subsequent to chemotherapy, or subsequent to chemotherapy.

71. The method of claim 37, further comprising a hydration step.

72. The method of any one of claims 37, 38, or 39, wherein said dithio-containing compound of the present invention is in a form suitable for administration by a method selected from the group consisting of: oral, injection, intra-cavitary, per rectum, and topical administration routes.

73. A method of augmenting the anti-cancer cytotoxic activity of chemotherapeutic agents by acting in an additive or synergistic cytotoxic manner with said chemotherapeutic agent or agents to increase intracellular oxidative stress within cancer cells, wherein said method comprises administering to a subject who has received one or more chemotherapeutic agents an effective amount of a pharmaceutically-acceptable form of a dithio-containing compound of the present invention, wherein said composition has an osmolarity of about 0.1- to about 5-times the osmolarity of the osmolarity of the normal plasma of said subject.

74. The method of claim 73, wherein said composition has an osmolarity of about 2- to about 4-times the osmolarity of the normal plasma of said subject.

75. The method of claim 73, wherein said composition has an osmolarity of about 3-times the osmolarity of the osmolarity of the normal plasma of said subject.

76. The method of any one of claims 73, 74, or 75, wherein said dithio-containing compound of the present invention is administered from about once a day to about once every five weeks.

77. The method of claim 73, wherein said dithio-containing compound of the present invention is administered from about once a day to about once every three weeks.

78. The method of claim 73, wherein said dithio-containing compound of the present invention is administered from about once a day to about once every two weeks.

79. The method of claim 73, wherein said dithio-containing compound of the present invention is administered from about once a day to about once every week.

80. The method of claim 73, wherein said dithio-containing compound of the present invention is administered in a time period which is selected from the group consisting of: about once every day, about once every two days, about once every three days, about once every four days, once about every five days, or about once every six days.

81. The method of any one of claims 73, 74, or 75, wherein said dithio-containing compound of the present invention is administered in a time period which is selected from the group consisting of: at least once in an approximately 24 hour period; at least once in an approximately 48 hour period; at least about once every three days; at least about once every four days; at least about once every five days; at least about once every six days; at least about once a week; at least about once every 1.5 weeks or less; at least about once every 2 weeks or less; at least about once every 2.5 weeks or less; at least about once every 3 weeks or less; at least about once every 3.5 weeks or less; at least about once every 4 weeks or less; at least about once every 5 weeks or less; at least once at any time interval between one day and five weeks; or at least once at a time interval of more than every five weeks.

82. The method of claim 73, wherein the augmentation of the anti-cancer activity of said chemotherapeutic agent or agents in said subject who has received one or more chemotherapeutic agents, and an effective amount of said dithio-containing compound of the present invention is caused by a prevention and/or reduction in the normal increase or responsiveness in the concentration and metabolism of glutathione, cysteine, and other physiological cellular thiols.

83. The method of claim 73, wherein the augmentation of the anti-cancer activity of said chemotherapeutic agent or agents in said subject who has received one or more chemotherapeutic agents, and an effective amount of said dithio-containing compound of the present invention is caused by thioredoxin inactivation, thereby increasing apoptotic sensitivity and decreasing mitogenic/cellular replication signaling.

84. The method of claim 73, wherein the augmentation of the anti-cancer activity of said chemotherapeutic agent or agents in said subject who has received one or more chemotherapeutic agents, and an effective amount of said dithio-containing compound of the present invention, e.g., 2,2′-dithio-bis-ethane sulfonate is caused by a key metabolite of 2,2′-dithio-bis-ethane sulfonate, 2-mercapto ethane sulfonate, which possesses intrinsic cytotoxic activity and causes apoptosis in tumors.

85. The method of claim 73, wherein the augmentation of the anti-cancer activity of said chemotherapeutic agent or agents in said subject who has received one or more chemotherapeutic agents, and an effective amount of said dithio-containing compound of the present invention is caused by reducing the oxidative potential or by compromising the anti-oxidative response of tumor cells and enhancing the oxidative biological state and oxidative damage in tumor cells exposed to chemotherapeutic agents, thereby enhancing the cytotoxic and apoptotic function of said chemotherapeutic agents.

86. The method of claim 73, wherein said one or more chemotherapeutic agents are selected from a group consisting of: fluropyrimidines, pyrimidine nucleosides, anti-folates, purine nucleosides, nucleotides, platinum analogs, anthracyclines/anthracenediones, epipodophyllotoxins, camptothecins, hormones and hormonal analogs, antihormones, enzymes, proteins, antibodies, vinca alkaloids, taxanes and taxane analogs, antimicrotubule agents, alkylating agents, epothilones, antimetabolites, topoisomerase inhibitors, aziridine-containing compounds, antiviral agents, monoclonal antibodies, proteins, peptides, enzymes, or cytostatic agents.

87. The method of claim 73, wherein the chemotherapeutic agent is a taxane analog.

88. The method of claim 73, wherein the chemotherapeutic agent is docetaxel.

89. The method of claim 73, wherein the chemotherapeutic agent is paclitaxel.

90. The method of claim 73, wherein the chemotherapeutic agent is a platinum analog.

91. The method of claim 73, wherein the chemotherapeutic agent is cisplatin.

92. The method of claim 73, wherein the chemotherapeutic agent is carboplatin.

93. The method of claim 73, wherein the chemotherapeutic agent is oxaliplatin.

94. The method of claim 73, wherein said method is utilized for the treatment of a subject with cancer.

95. The method of claim 94, wherein said subject is human.

96. The method of claim 94 or claim 95, wherein said method is utilized for treating any one or more cancers selected from the group consisting of: ovary, breast, lung, esophagus, bladder, stomach, pancreas, liver (e.g., bile ducts, gall bladder, and Ampulla of Vater), testes, germ cell, bone, cartilage, head, neck, oral mucosa, colorectal area, anus, kidney, uroepithelium, central nervous system, prostate, endometrium, cervix, uterus, fallopian tube, peripheral nervous system, and various other cancers including melanoma, mesothelioma, myeloma, lymphoma, leukemia, and Kaposi's sarcoma.

97. The method of any one of claims 73, 74, or 75, wherein said method further comprises the prevention, reduction or mitigation of one or more toxicities associated with administration of said chemotherapeutic agent or agents.

98. The method of any one of claims 73, 74, or 75, wherein the augmentation of the anti-cancer activity of the chemotherapeutic agent allows said chemotherapeutic agent to be administered at a lower dose, while still achieving the same degree of clinical efficacy as would be obtained with the administration of a higher dose.

99. The method of any one of claims 73, 74, or 75, wherein said dithio-containing compound is disodium 2,2′-dithio-bis-ethane sulfonate.

100. The method of claim 73, wherein said dithio-containing compound of the present invention is a pharmaceutically-acceptable salt.

101. The method of claim 100, wherein said salt is a disodium salt.

102. The method of claim 100, wherein said salt is selected from the group consisting of: a monosodium salt, a sodium potassium salt, a dipotassium salt, a calcium salt, a magnesium salt, a manganese salt, a monopotassium salt, or an ammonium salt.

103. The method of claim 73, further comprising administering a pre-therapy treatment at least one day prior to chemotherapy, prior to each chemotherapy treatment, immediately prior to each chemotherapy treatment, concomitantly with or simultaneously during chemotherapy treatment, immediately subsequent to chemotherapy, or subsequent to chemotherapy.

104. The method of claim 73, further comprising a hydration step.

105. The method of any one of claims 73, 74, or 75, wherein said dithio-containing compound of the present invention is in a form suitable for administration by a method selected from the group consisting of: oral, injection, intra-cavitary, per rectum, and topical administration routes.

106. A method of augmenting the anti-cancer cytotoxic activity of chemotherapeutic agents by acting in an additive or synergistic cytotoxic manner with said chemotherapeutic agent or agents to increase intracellular oxidative stress within cancer cells, wherein said method comprises administering to a subject who has received one or more chemotherapeutic agents an effective amount of a pharmaceutically-acceptable form of said dithio-containing compound of the present invention at a rate of about 0.1 g/min to about 4.6 g/min, at a total dose of about 4 g/m² to about 41 g/m².

107. The method of claim 106, wherein said dithio-containing compound of the present invention is administered at a rate of about 0.2 g/min. to about 2.0 g/min.

108. The method of claim 106, wherein said dithio-containing compound of the present invention is administered at a rate of about 0.7 g/min.

109. The method of any one of claims 106, 107, or 108, wherein said dithio-containing compound of the present invention is administered over a period of about 45 minutes.

110. The method of any one of claims 106, 107, or 108, wherein said dithio-containing compound of the present invention is administered at a concentration of about 100 mg/mL.

111. The method of any one of claims 106, 107, or 108, wherein said dithio-containing compound of the present invention is administered over a period of about 45 minutes, at a concentration of about 100 mg/mL.

112. The method of any one of claims 106, 107, or 108, wherein said dithio-containing compound of the present invention is administered from about once a day to about once every five weeks.

113. The method of claim 106, wherein said dithio-containing compound of the present invention is administered from about once a day to about once every three weeks.

114. The method of claim 106, wherein said dithio-containing compound of the present invention is administered from about once a day to about once every two weeks.

115. The method of claim 106, wherein said dithio-containing compound of the present invention is administered from about once a day to about once every week.

116. The method of claim 106, wherein said dithio-containing compound of the present invention is administered in a time period which is selected from the group consisting of: about once every day, about once every two days, about once every three days, about once every four days, once about every five days, or about once every six days.

117. The method of any one of claims 106, 107, or 108, wherein said dithio-containing compound of the present invention is administered in a time period which is selected from the group consisting of: at least once in an approximately 24 hour period; at least once in an approximately 48 hour period; at least about once every three days; at least about once every four days; at least about once every five days; at least about once every six days; at least about once a week; at least about once every 1.5 weeks or less; at least about once every 2 weeks or less; at least about once every 2.5 weeks or less; at least about once every 3 weeks or less; at least about once every 3.5 weeks or less; at least about once every 4 weeks or less; at least about once every 5 weeks or less; at least once at any time interval between one day and five weeks; or at least once at a time interval of more than every five weeks.

118. The method of claim 106, wherein the augmentation of the anti-cancer activity of said chemotherapeutic agent or agents in said subject who has received one or more chemotherapeutic agents, and an effective amount of said dithio-containing compound of the present invention is caused by a prevention and/or reduction in the normal increase or responsiveness in the concentration and metabolism of glutathione, cysteine, and other physiological cellular thiols.

119. The method of claim 106, wherein the augmentation of the anti-cancer activity of said chemotherapeutic agent or agents in said subject who has received one or more chemotherapeutic agents, and an effective amount of said dithio-containing compound of the present invention is caused by thioredoxin inactivation, thereby increasing apoptotic sensitivity and decreasing mitogenic/cellular replication signaling.

120. The method of claim 106, wherein the augmentation of the anti-cancer activity of said chemotherapeutic agent or agents in said subject who has received one or more chemotherapeutic agents, and an effective amount of said dithio-containing compound of the present invention, e.g., 2,2′-dithio-bis-ethane sulfonate is caused by a key metabolite of 2,2′-dithio-bis-ethane sulfonate, 2-mercapto ethane sulfonate, which possesses intrinsic cytotoxic activity and causes apoptosis in tumors.

121. The method of claim 106, wherein the augmentation of the anti-cancer activity of said chemotherapeutic agent or agents in said subject who has received one or more chemotherapeutic agents, and an effective amount of said dithio-containing compound of the present invention is caused by reducing the oxidative potential or by compromising the anti-oxidative response of tumor cells and enhancing the oxidative biological state and oxidative damage in tumor cells exposed to chemotherapeutic agents, thereby enhancing the cytotoxic and apoptotic function of said chemotherapeutic agents.

122. The method of claim 106, wherein said one or more chemotherapeutic agents are selected from a group consisting of: fluropyrimidines, pyrimidine nucleosides, anti-folates, purine nucleosides, nucleotides, platinum analogs, anthracyclines/anthracenediones, epipodophyllotoxins, camptothecins, hormones and hormonal analogs, antihormones, enzymes, proteins, antibodies, vinca alkaloids, taxanes and taxane analogs, antimicrotubule agents, alkylating agents, epothilones, antimetabolites, topoisomerase inhibitors, aziridine-containing compounds, antiviral agents, monoclonal antibodies, proteins, peptides, enzymes, or cytostatic agents.

123. The method of claim 106, wherein the chemotherapeutic agent is a taxane analog.

124. The method of claim 106, wherein the chemotherapeutic agent is docetaxel.

125. The method of claim 106, wherein the chemotherapeutic agent is paclitaxel.

126. The method of claim 106, wherein the chemotherapeutic agent is a platinum analog.

127. The method of claim 106, wherein the chemotherapeutic agent is cisplatin.

128. The method of claim 106, wherein the chemotherapeutic agent is carboplatin.

129. The method of claim 106, wherein the chemotherapeutic agent is oxaliplatin.

130. The method of claim 106, wherein said method is utilized for the treatment of a subject with cancer.

131. The method of claim 130, wherein said subject is human.

132. The method of claim 130 or claim 131, wherein said method is utilized for treating any one or more cancers selected from the group consisting of: ovary, breast, lung, esophagus, bladder, stomach, pancreas, liver (e.g., bile ducts, gall bladder, and Ampulla of Vater), testes, germ cell, bone, cartilage, head, neck, oral mucosa, colorectal area, anus, kidney, uroepithelium, central nervous system, prostate, endometrium, cervix, uterus, fallopian tube, peripheral nervous system, and various other cancers including melanoma, mesothelioma, myeloma, lymphoma, leukemia, and Kaposi's sarcoma.

133. The method of any one of claims 106, 107, or 108, wherein said method further comprises the prevention, reduction or mitigation of one or more toxicities associated with administration of said chemotherapeutic agent or agents.

134. The method of any one of claims 106, 107, or 108, wherein the augmentation of the anti-cancer activity of the chemotherapeutic agent allows said chemotherapeutic agent to be administered at a lower dose, while still achieving the same degree of clinical efficacy as would be obtained with the administration of a higher dose.

135. The method of any one of claims 106, 107, or 108, wherein said dithio-containing compound is disodium 2,2′-dithio-bis-ethane sulfonate.

136. The method of claim 106, wherein said dithio-containing compound of the present invention is a pharmaceutically-acceptable salt.

137. The method of claim 136, wherein said salt is a disodium salt.

138. The method of claim 136, wherein said salt is selected from the group consisting of: a monosodium salt, a sodium potassium salt, a dipotassium salt, a calcium salt, a magnesium salt, a manganese salt, a monopotassium salt, or an ammonium salt.

139. The method of claim 106, further comprising administering a pre-therapy treatment at least one day prior to chemotherapy, prior to each chemotherapy treatment, immediately prior to each chemotherapy treatment, concomitantly with or simultaneously during chemotherapy treatment, immediately subsequent to chemotherapy, or subsequent to chemotherapy.

140. The method of claim 106, further comprising a hydration step.

141. The method of any one of claims 106, 107, or 108, wherein said dithio-containing compound of the present invention is in a form suitable for administration by a method selected from the group consisting of: oral, injection, intra-cavitary, per rectum, and topical administration routes.

142. A method of augmenting the anti-cancer activity of chemotherapeutic agents by acting in an additive or synergistic manner with said chemotherapeutic agent or agents to alter the intracellular oxidative/reduction potential within cancer cells, wherein said method comprises administering to a subject with cancer an effective amount of a pharmaceutically-acceptable form of a dithio-containing compound of the present invention.

143. A method of selectively altering the intracellular oxidative/reduction potential within cancer cells versus normal, non-cancerous cells in order to have a therapeutic effect on a subject with cancer, wherein said method comprises administering to said subject an effective amount of a pharmaceutically-acceptable form of a dithio-containing compound of the present invention.

144. A method of selectively altering the intracellular oxidative/reduction potential within cancer cells versus normal, non-cancerous cells in order to selectively sensitize the cancer cells of a subject with cancer in order to have a therapeutic effect on said subject, wherein said method comprises administering to said subject with cancer an effective amount of a pharmaceutically-acceptable form of a dithio-containing compound of the present invention.

145. A method of augmenting the anti-cancer activity of chemotherapeutic agents by acting in an additive or synergistic manner with said chemotherapeutic agent or agents to alter the intracellular oxidative/reduction potential within cancer cells in a subject with cancer, wherein said method comprises administering to a subject who has received, is receiving, or will subsequently receive one or more chemotherapeutic agents, an effective amount of a pharmaceutically-acceptable form of a dithio-containing compound of the present invention.

146. A method of selectively altering the intracellular oxidative/reduction potential within cancer cells versus normal, non-cancerous cells in order to have a therapeutic effect on a subject with cancer, wherein said method comprises administering to said subject with cancer who has received, is receiving, or will subsequently receive one or more chemotherapeutic agents, an effective amount of a pharmaceutically-acceptable form of a dithio-containing compound of the present invention.

147. A method of selectively altering the intracellular oxidative/reduction potential within cancer cells versus normal, non-cancerous cells in order selectively sensitize the cancer cells of a subject with cancer in order to have a therapeutic effect on said subject, wherein said method comprises administering to said subject with cancer who has received, is receiving, or will subsequently receive one or more chemotherapeutic agents, an effective amount of a pharmaceutically-acceptable form of a dithio-containing compound of the present invention.

148. A method of augmenting the anti-cancer activity of chemotherapeutic agents by acting in an additive or synergistic manner with said chemotherapeutic agent or agents to alter the intracellular oxidative/reduction potential within cancer cells in a subject with cancer, wherein said method comprises administering to a subject who has received, is receiving, or will subsequently receive one or more chemotherapeutic agents, an effective amount of a pharmaceutically-acceptable form of a dithio-containing compound of the present invention at a rate of about 0.1 g/min. to about 2.0 g/min.

149. A method of selectively altering the intracellular oxidative/reduction potential within cancer cells versus normal, non-cancerous cells in order to have a therapeutic effect on a subject with cancer, wherein said method comprises administering to said subject with cancer who has received, is receiving, or will subsequently receive one or more chemotherapeutic agents, an effective amount of a pharmaceutically-acceptable form of a dithio-containing compound of the present invention at a rate of about 0.1 g/min. to about 2.0 g/min.

150. A method of selectively altering the intracellular oxidative/reduction potential within cancer cells versus normal, non-cancerous cells in order selectively sensitize the cancer cells of a subject with cancer in order to have a therapeutic effect on said subject, wherein said method comprises administering to said subject with cancer who has received, is receiving, or will subsequently receive one or more chemotherapeutic agents, an effective amount of a pharmaceutically-acceptable form of a dithio-containing compound of the present invention at a rate of about 0.1 g/min. to about 2.0 g/min.

151. The method of any one of claims 142 to 150, wherein the dithio-containing compound is disodium 2,2′-dithio-bis-ethane sulfonate.

152. The method of any one of claims 142 to 151, wherein said one or more chemotherapeutic agents are selected from the group consisting of: fluropyrimidines, pyrimidine nucleosides, anti-folates, purine nucleosides, nucleotides, platinum analogs, anthracyclines/anthracenediones, epipodophyllotoxins, camptothecins, hormones and hormonal analogs, antihormones, enzymes, proteins, antibodies, vinca alkaloids, taxanes and taxane analogs, antimicrotubule agents, alkylating agents, epothilones, antimetabolites, topoisomerase inhibitors, aziridine-containing compounds, antiviral agents, monoclonal antibodies, proteins, peptides, enzymes, or cytostatic agents.

153. The method of any one of claims 142 to 150, wherein the one or more cancers are selected from the group consisting of: ovary, breast, lung, esophagus, bladder, stomach, pancreas, liver (e.g., bile ducts, gall bladder, and Ampulla of Vater), testes, germ cell, bone, cartilage, head, neck, oral mucosa, colorectal area, anus, kidney, uroepithelium, central nervous system, prostate, endometrium, cervix, uterus, fallopian tube, peripheral nervous system, and various other cancers including melanoma, mesothelioma, myeloma, lymphoma, leukemia, and Kaposi's sarcoma.

154. A medical device which possesses the ability to administer to a subject with cancer a composition comprising a dithio-containing compound of the present invention which possesses the ability to selectively alter the intracellular oxidative/reduction potential within cancer cells versus normal, non-cancerous cells in order to have a therapeutic effect on said subject, wherein said device utilizes the method set forth in any one of claims 1, 37, 73, 106, 142, or 142 to 150.

155. The medical device of claim 154, wherein said device is an implantable infusion device.

156. The medical device of claim 155, wherein said implantable infusion device is a passive infusion device.

157. The medical device of claim 155, wherein said implantable infusion device is an active infusion device.

158. The device of any one of claim 154-157, wherein said composition further comprises one or more chemotherapeutic agents selected from the group consisting of: fluropyrimidines, pyrimidine nucleosides, anti-folates, purine nucleosides, nucleotides, platinum analogs, anthracyclines/anthracenediones, epipodophyllotoxins, camptothecins, hormones and hormonal analogs, antihormones, enzymes, proteins, antibodies, vinca alkaloids, taxanes and taxane analogs, antimicrotubule agents, alkylating agents, epothilones, antimetabolites, topoisomerase inhibitors, aziridine-containing compounds, antiviral agents, monoclonal antibodies, proteins, peptides, enzymes, or cytostatic agents.

159. The medical device of claim 154, wherein said dithio-containing compound is disodium 2,2′-dithio-bis-ethane sulfonate.

160. A medical device which possesses the ability to administer to a subject in need thereof a composition comprising a dithio-containing compound of the present invention, wherein said composition is administered to said subject at a rate of about 0.1 g/min. to about 4.6 g/mL/min., for a total dose of about 4 g/m² to about 41 g/m².

161. The device of claim 160, wherein said device is an implantable infusion device.

162. The device of claim 161, wherein said implantable infusion device is a passive infusion device.

163. The device of claim 161, wherein said implantable infusion device is an active infusion device.

164. The device of any one of claim 160-163, wherein said composition further comprises one or more chemotherapeutic agents selected from the group consisting of: fluropyrimidines, pyrimidine nucleosides, anti-folates, purine nucleosides, nucleotides, platinum analogs, anthracyclines/anthracenediones, epipodophyllotoxins, camptothecins, hormones and hormonal analogs, antihormones, enzymes, proteins, antibodies, vinca alkaloids, taxanes and taxane analogs, antimicrotubule agents, alkylating agents, epothilones, antimetabolites, topoisomerase inhibitors, aziridine-containing compounds, antiviral agents, monoclonal antibodies, proteins, peptides, enzymes, or cytostatic agents.

165. The method of claim 160, wherein said dithio-containing compound is disodium 2,2′-dithio-bis-ethane sulfonate.

166. A medical device which possesses the ability to administer to a subject in need thereof a composition comprising a dithio-containing compound of the present invention, wherein said composition is administered to said subject at a rate of about 1 g/mULmin. to about 50 g/mL/min.

167. The medical device of claim 166, wherein said device is an implantable infusion device.

168. The medical device of claim 167, wherein said implantable infusion device is a passive infusion device.

169. The device of claim 167, wherein said implantable infusion device is an active infusion device.

170. The device of any one of claims 166-169, wherein said composition further comprises one or more chemotherapeutic agents selected from the group consisting of: fluropyrimidines, pyrimidine nucleosides, anti-folates, purine nucleosides, nucleotides, platinum analogs, anthracyclines/anthracenediones, epipodophyllotoxins, camptothecins, hormones and hormonal analogs, antihormones, enzymes, proteins, antibodies, vinca alkaloids, taxanes and taxane analogs, antimicrotubule agents, alkylating agents, epothilones, antimetabolites, topoisomerase inhibitors, aziridine-containing compounds, antiviral agents, monoclonal antibodies, proteins, peptides, enzymes, or cytostatic agents.

171. The method of claim 166, wherein said dithio-containing compound is disodium 2,2′-dithio-bis-ethane sulfonate.

172. A medical device which possesses the ability to administer to a subject in need thereof a composition comprising a dithio-containing compound of the present invention, wherein said composition is administered to said subject at a rate of about 0.1- to about 5-times the osmolarity of the normal range of plasma osmolarity.

173. The device of claim 172, wherein said device is an implantable infusion device.

174. The device of claim 173, wherein said implantable infusion device is a passive infusion device.

175. The device of claim 173, wherein said implantable infusion device is an active infusion device.

176. The device of any one of claims 172-175, wherein said composition further comprises one or more chemotherapeutic agents selected from the group consisting of: fluropyrimidines, pyrimidine nucleosides, anti-folates, purine nucleosides, nucleotides, platinum analogs, anthracyclines/anthracenediones, epipodophyllotoxins, camptothecins, hormones and hormonal analogs, antihormones, enzymes, proteins, antibodies, vinca alkaloids, taxanes and taxane analogs, antimicrotubule agents, alkylating agents, epothilones, antimetabolites, topoisomerase inhibitors, aziridine-containing compounds, antiviral agents, monoclonal antibodies, proteins, peptides, enzymes, or cytostatic agents.

177. The method of claim 172, wherein said dithio-containing compound is disodium 2,2′-dithio-bis-ethane sulfonate.
